(19) United States
(12) Patent Application Publication
(10) Pub. No.: US 2012/0245683 A1
(43) Pub. Date: Sep. 27, 2012

(54) CORNEAL IMPLANT FOR REFRACTIVE CORRECTION

(75) Inventors: Bruce A. Christie, Claremont, CA (US); Thomas Silvestrini, Alamo, CA (US)

(73) Assignee: ACUFOCUS, INC., Irvine, CA (US)

(21) Appl. No.: 13/513,699

(22) PCT Filed: Dec. 3, 2010
(86) PCT No.: PCT/US10/58879

§ 371 Date: Jun. 4, 2012

Related U.S. Application Data
(60) Provisional application No. 61/266,853, filed on Dec. 4, 2009.

(51) Int. Cl. A61F 2/14

(52) U.S. Cl. 623/5.11

(57) ABSTRACT

A corneal implant adapted for implantation between layers of a cornea to focus an image on a retina of an eye includes an inlay, an outer perimeter, and a clear central region capable of refracting light to compensate for a refractive error of an eye. The inlay also has an annular opaque region comprising a plurality of holes or otherwise being adapted to transport nutrients. The annular opaque region extends from the outer circumference of the inlay to the clear central portion. The opaque region extends over a minority of the surface area of the implant. The anterior and posterior surfaces of the inlay are configured to abut adjacent layers of the cornea.

### CROSS-REFERENCE TO RELATED APPLICATIONS

This application claims the benefit of U.S. Provisional Application No. 61/266,853, filed Dec. 4, 2009, the entirety of which is hereby incorporated by reference.

### BACKGROUND OF THE INVENTION

1. Field of the Invention

This application is directed to devices that can be deployed within a human cornea to compensate for at least one of refractive error and loss of accommodation, and to related methods.

2. Description of the Related Art

When a human eye focuses on objects, light rays from the object converge at the retina, located at the back of the eye. Such convergence of light rays is due to accommodation of the crystalline lens and refraction at the anterior surface of the cornea and at the interfaces between the cornea, the aqueous humor, the crystalline lens, and the vitreous humor. In the normal eye, light rays from a distant object which enter the eye parallel to an optical axis of the eye are focused (caused to converge) directly at the retina. Convergence of these rays at the retina results in a clear image of the distant object. Light rays from near objects reach the eye at a divergent angle. All other variables remaining constant, the diverging light rays would converge at a point behind the retina, resulting in an unfocused image of near objects. In the normal eye, the lens deforms to cause the point of convergence of the light to be moved forward to the retina so that the near object image is focused on the retina.

Unfortunately, several common defects in the eye impair the ability of the eye to focus an image as discussed above. For example, ametropia includes a variety of refractive defects in which images are not focused at the retina. Ametropia can be caused by a discrepancy between the refractive power of the eye and the dimensions of the eye. Forms of ametropia include myopia, hyperopia and astigmatism.

Myopia, also known as nearsightedness, is caused by a mismatch between the refractive power of the eye and the dimensions of the eye that results in light rays entering the eye parallel to the optical axis being focused in front of the retina. On the other hand, the diverging light rays from near objects converge at the retina with little or no intraocular lens deformation (known as “accommodation”) and thus are in focus. With full accommodation of the lens, the myopic eye can focus light rays from objects that are very close to the eye, hence the term nearsightedness.

Hyperopia, also known as farsightedness, also can be caused by a mismatch between the refractive power and the dimensions of the eye that results in light rays entering the eye parallel to the optical axis being focused behind the retina. Accommodation enables the eye to bring the image of the far object into sharp focus on the retina. For near objects, however, the hyperopic eye focuses the diverging light rays which enter the eye at a point far behind the retina. Due to a limit in the amount of deformation of the intraocular lens, however, the point of focus for near objects still falls behind the retina, resulting in an unfocused image. The nearest point of distinct vision in such an eye with full accommodation of the crystalline lens is farther removed from the eye, hence the term farsightedness.

Astigmatism is a condition that occurs when parallel rays of light do not focus to a single point within the eye, but rather have a variable focus due to the fact that the cornea is more curved in one meridian than in another. In this configuration, the eye refracts light rays in different meridians at different distances. Some degree of astigmatism is normal, but where it is pronounced, the astigmatism may require correction.

Farsightedness has traditionally been treated with positive power spectacles, or glasses, or contact lenses, which converge the light rays somewhat before they reach the eye, improving near vision. Nearsightedness has traditionally been treated with negative power spectacles or contact lenses, which diverge the light rays somewhat before they reach the eye, improving distance vision. Astigmatism has traditionally been treated with cylindrical spectacles or contact lenses, which have different radii of curvature in different planes to focus parallel rays of light on a single point within the eye.

While the foregoing treatments of poor vision due to refractive error or mismatch between refraction and other eye dimension work for most patient, they are generally inconvenient. For example, glasses can be lost or damaged when removed, e.g., for sleeping or to be exchanged for sunglasses. Similarly, contact lenses are inconvenient in that they need to be kept clean and periodically replaced. Some patients find glasses and contact lenses uncomfortable and would prefer not to wear them. While the use of these devices recently has been reduced by the introduction of laser surgery (e.g., LASIK and similar procedures), many patients are uncomfortable with these procedures because they physically change the eye (e.g. remove tissue from the eye) and thus are irreversible.

### SUMMARY OF THE INVENTIONS

Because of the disadvantages of these various prior art approaches, it is desirable to provide an improved surgical method and associated apparatus for correcting refractive defects of the eye using an intracorneal implant. It is desirable that such a method provide a permanent, but reversible, correction of vision defects without substantial trauma to the corneal tissue.

There is provided in accordance with one aspect of the present invention, an ocular device suitable for implantation between layers of a cornea of an eye. The ocular device includes an implant body having a first zone with a first transmissivity for alignment with an optical axis and a second zone having a lower transmissivity, wherein the second zone at least partially surrounds the first zone. The first zone has a water content of at least about 25%, alternatively at least about 30%, alternatively at least about 35%, alternatively no more than 55% when immersed in normal saline at standard temperature and pressure (STP). The second zone has a water content of less than about 10% when immersed in normal saline at STP.

In certain embodiments, the first zone may comprise a transparent region, for example having a transmissivity of at least 85%, and the second zone may comprise an opaque region, for example having a transmissivity of no more than about 15% in the visible range.

In an alternative embodiment, a corneal implant adapted for positioning between first and second layers of a cornea is provided. The corneal implant includes an annular mask portion having a transmission in the visible range of no more than about 20% and a central lens portion having a transmission in the visible range of at least about 80%. The central lens portion has a water content of at least about 25% and the mask portion has a water content of no more than 10% when immersed in normal saline at equilibrium at STP.

In an alternative embodiment, an implant for positioning across an optical axis of a patient's eye is provided. The implant includes an implant body having a first zone comprising a material with a transmission of at least about 80% in the visible range and a second zone surrounding the first zone. The second zone comprises a material with a transmission of no more than about 20% in the visible range. The first material is configured to expand in an aqueous environment at least about 25% by volume and the second material is configured to expand in an aqueous environment by between about 0-10% by volume.

In an alternative embodiment, a corneal implant adapted for positioning between first and second layers of a cornea is provided. The implant includes an annular mask portion comprising a first material having a transmission in the visible range of no more than about 25% and a central lens portion comprising a second material having a transmission in the visible range of at least about 75%. The lens portion has a water content of at least about 25% and expands by at least about 25% by volume and the mask portion has a water content of no more than about 10% when immersed in normal saline at equilibrium at STP.

In an alternative embodiment, a corneal implant adapted for positioning between first and second layers of a cornea is provided. The implant includes an annular mask portion having a transmission in the visible range of no more than about 20% and a central lens portion comprising having a transmission in the visible range of at least about 80%. The annular mask portion has a glucose transportability of at least 50%, for example as much as 95%. For example, the annular mask portion can maintain at least 50% of glucose level that would be present if the annular mask portion not present by transporting glucose across the annular mask portion. The corneal implant with the central lens portion has a glucose transportability of at least about 50%, and in some cases about 68% or more. In some embodiments, glucose transportability is at least about 75% or more.

In an alternative embodiment, a corneal implant adapted for positioning between first and second layers of a cornea is provided. The implant includes an annular mask portion having a transmission in the visible range of no more than about 20% and a central lens portion having a transmission in the visible range of at least about 80%. The expansion ratio of the lens to the mask in an aqueous environment is at least about 3:1.

In an alternative embodiment, an ocular device suitable for implantation between layers of a cornea of an eye is provided. The ocular device includes a lens body having an outer perimeter and an anterior surface that extends to the outer perimeter. The anterior surface is configured to reside adjacent a first corneal layer. The lens body also has a posterior surface that extends to the outer perimeter. The posterior surface is configured to reside adjacent a second corneal layer. A transparent region is located at least partially within the outer perimeter and is capable of refracting light to compensate for a refractive error of an eye. A nontransmissive region, which can be an opaque region, can extend between the outer perimeter and the transparent region. The lens body also has a plurality of recesses that extend from at least one of the anterior and posterior surfaces. The recesses can be confined to the nontransmissive region. A transverse dimension (e.g., a diameter) of the nontransmissive portion is greater than a transverse dimension (e.g., a diameter) of the transparent region. In some embodiments, the nontransmissive portion comprises an annular structure with a width that is less than the transverse dimension (e.g., diameter) of the transparent region.

In an alternative embodiment, there is provided a corneal implant adapted for implantation between layers of a cornea to help the eye focus an image on a retina of an eye. The corneal implant includes a lens body having anterior and posterior surfaces and an outer circumference. The lens body also has a clear central region capable of refracting light to compensate for a refractive error of an eye and an annular opaque region comprising a plurality of holes. The annular opaque region extends from the outer circumference of the lens body to the clear central portion. The opaque region extends over a minority of the surface area of the implant. The anterior and posterior surfaces of the lens body are configured to abut adjacent layers of the cornea.

In an alternative embodiment, there is provided an ocular device suitable for implantation between layers of a cornea of an eye. The ocular device includes a nontransmisive portion and a transparent portion. The nontransmissive portion has a plurality of recesses that extend from at least one of an anterior surface and a posterior surface. The nontransmissive portion extends between an outer periphery and an inner periphery. The transparent portion is capable of refracting light to compensate for a refractive error of an eye. The transparent portion is configured to provide secure engagement with the inner periphery of the opaque portion. For example, the transparent portion can be configured to expand into engagement with the inner periphery of the opaque portion. In one embodiment, the transparent portion has a transverse dimension that is greater than that required to produce a pinhole effect.

In an alternative embodiment, a method is provided for treating a patient. An ocular device is provided that comprises an annular mask portion having a transmission in the visible range of no more than about 20% and a central lens portion having a transmission in the visible range of at least about 80%. The lens portion has a water content of at least about 25% and the mask portion has a water content of no more than about 10% when immersed in normal saline at equilibrium at STP. The ocular device is positioned such that an optical axis of the patient intersects the central lens portion.

In another embodiment, a method of making an optical implant is provided. In the method, a lens is formed of a first material that includes a network of absorbent polymer chains and a diluent. The diluent is absorbed by the network of polymer chains. The diluent is exchanged with or replaced by with a liquid, e.g., saline or water, when in contact therewith. Diluent exchange permits the lens to have approximately the same volume when formed and when used, e.g., in an aqueous environment.

In another method, a lens formed by diluent exchange can be coupled with an annular mask portion or a nontransmissive portion comprised of a second material. The second material is different from the first material.

For purposes of summarizing the invention, certain aspects, advantages and novel features of the invention are described herein. It is to be understood that not all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

Further features and advantages of the present invention will become apparent from the detailed description of preferred embodiments which follows, when considered together with the attached drawings and claims.

FIG. 1 is a schematic representation of a horizontal cross-section of the eye.

FIG. 2 is a schematic illustration of the anterior portion of the eye showing the various layers of the cornea.

FIG. 3 is a schematic representation showing how light from an object can be focused on the retina of a normal eye.

FIG. 4 is a schematic representation of how light from an object does not focus on the retina of a myopic eye.

FIG. 5 is a schematic representation of an ocular device having a refractive power implanted in a myopic eye, the ocular device focusing light from an object on the retina of the myopic eye.

FIG. 6A is a top plan view of one embodiment of an ocular device that can be used to compensate for refractive error.

FIG. 6B is a cross-sectional view of the ocular device of FIG. 6A implanted in the cornea showing tissue being drawn into the recesses of the device.

FIG. 7A is a cross-sectional view of the ocular device of FIG. 6A having a negative power lens.

FIG. 7B is a cross-sectional view of an alternative embodiment of an ocular device having a positive power lens that can compensate for refractive error.

FIG. 7C is a cross-sectional view of an alternative embodiment of an ocular device having a positive lens that can be used to compensate for refractive error.

FIG. 7D is a cross-sectional view of an alternative embodiment of an ocular device having a hydrogel inlay.

FIG. 8 is a schematic representation of how divergent light rays from a near object does not focus on the retina of a presbyopic eye.

FIG. 9 is a schematic representation of light transmitted through a presbyopic eye having implanted therein an ocular device with both pin-hole (or stenopaeic) correction and refractive correction.

FIG. 10A is top plan view of an alternative embodiment of an ocular device that can be used to compensate for refractive error and for a decrease in accommodation.

FIG. 10B is a cross-sectional view of the ocular device of FIG. 10A implanted in the cornea.

FIG. 10C is a cross-sectional view of a portion of an ocular device configured to provide a mechanical coupling of a transmissive zone with a nontransmissive zone.

FIG. 11 is top plan view of an alternative embodiment of an ocular device including a locator structure.

FIG. 12 is top plan view of an alternative embodiment of an ocular device including a locator structure.

FIGS. 13A-13B illustrate a technique for implanting an ocular device.

FIGS. 14A-14E illustrate an alternative technique for implanting an ocular device.

FIG. 15 illustrates a technique for making an ocular device.

FIGS. 16 illustrate another technique for making an ocular device.

FIGS. 17A-C illustrate another technique for making an ocular device.

FIGS. 18A-D illustrate alternative embodiments of an ocular device that include a rib structure.

### DETAILED DESCRIPTION OF THE PREFERRED EMBODIMENTS

This application is directed to devices and methods that compensate for refractive error of a patient's eye. In some embodiments discussed below, a device that is capable of compensating for such refractive errors is an intra corneal lens. The corneal lenses discussed herein can be deployed within the cornea using a variety of techniques and, as such, the term “inlay” or “corneal inlay” is sometimes used. Other ocular devices and corneal lenses that are suitable for compensating for refractive error or otherwise improving a patient's vision can be placed on or in the cornea, e.g., on or in the epithelium of the eye.

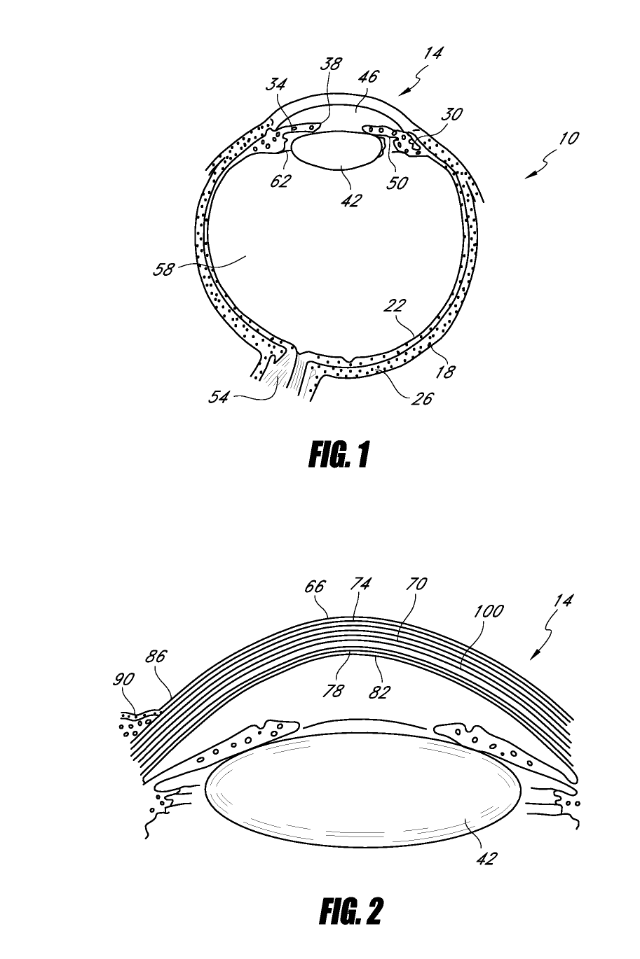

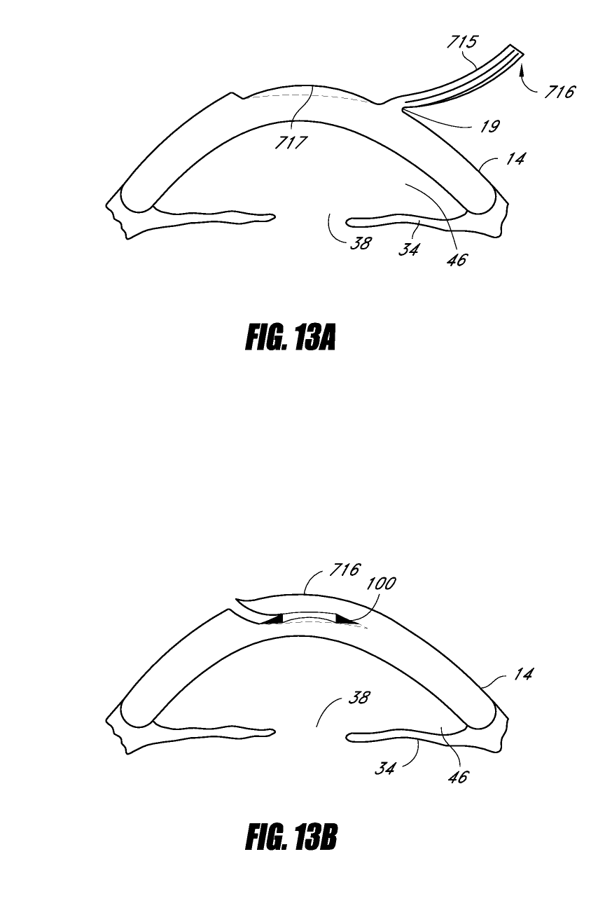

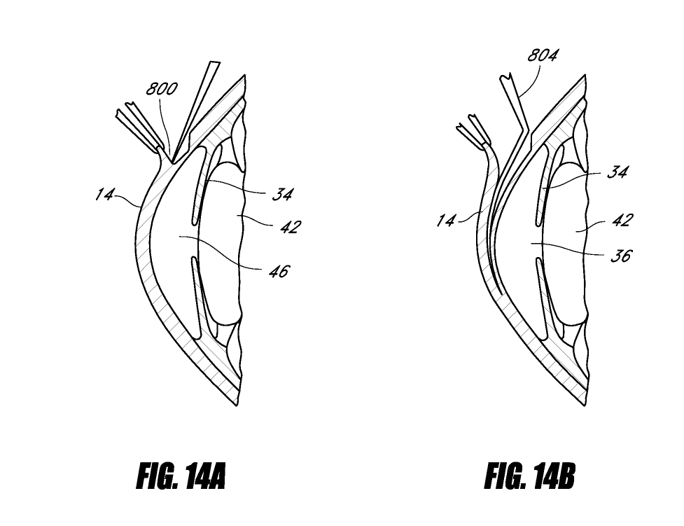

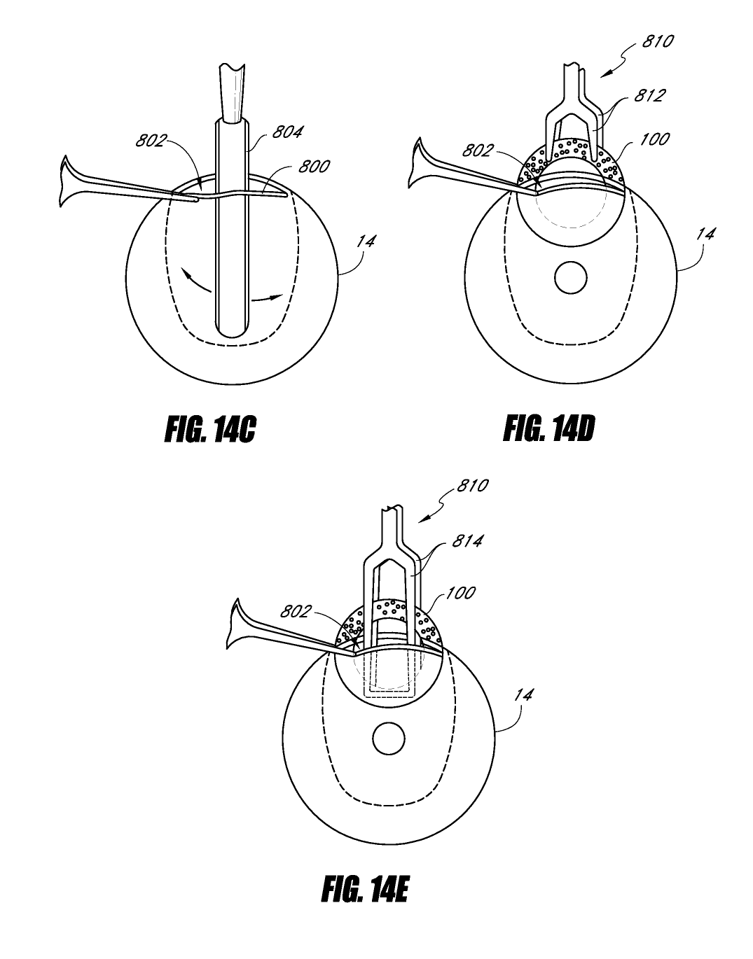

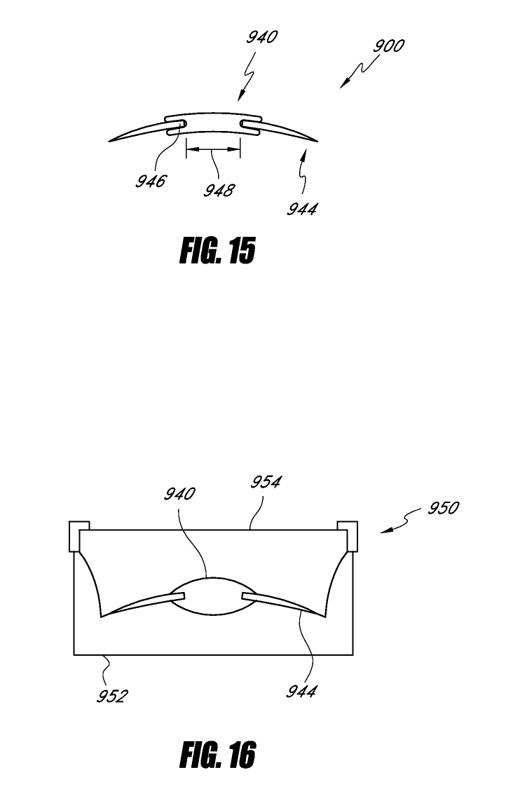

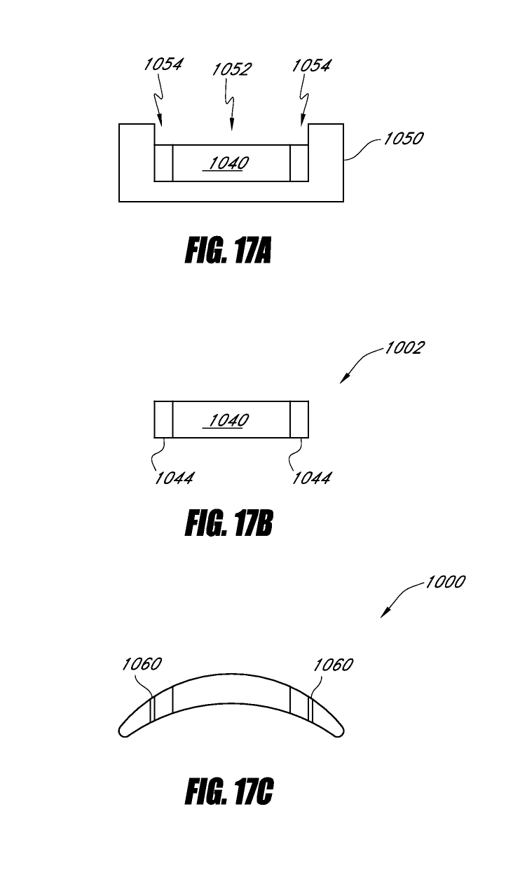

Prior to discussing the details of various embodiments of such an ocular device, the effects of refractive errors are set forth in connection with FIGS. 1-4. Thereafter, a variety of embodiments that compensate for refractive error, some of which additionally provide increased depth of field, will be discussed in connection with FIGS. 5-12. Various techniques for implanting an ocular device within the cornea can be employed, such as those discussed in connection with FIGS. 13A-14C. Various techniques for making ocular devices are discussed in connection with FIGS. 15-17C. Finally, additional embodiments of ocular devices with rib structures are discussed in connection with FIGS. 18A-D.

### I. Compensating for Refractive Errors in Human Vision

FIG. 1 shows a horizontal section of an eyeball or eye 10. The eye 10 includes a cornea 14, which is an anterior bulged spherical portion of the eye 10, and a sclera 18 enclosing transparent media through which the light passes to reach the retina 22. The retina 22 includes light sensitive tissue and is located at the back of the eye 10. The sclera 18 is a fibrous protective portion and constitutes approximately the posterior five-sixths of the eye 10. The sclera 18 is white and opaque and the visible portion of the sclera is sometimes referred to as the “white” of the eye. The anterior one-sixth of the eye 10 is the cornea 14.

An interior covering of the eye 10 is vascular and nutritive in function and includes the choroid 26, the ciliary body 30, and the iris 34. This interior covering maintains the retina 22. The ciliary body 30 supports a lens 42 and is involved in accommodation, as discussed below. The iris 34 is located in an anterior portion of the interior covering of the eye 10 and is arranged in a frontal plane. The iris 34 includes a thin circular disc that is perforated near its center by a circular aperture called the pupil 38. The iris 34 is analogous to the diaphragm of a camera in that the size of the pupil 38 varies to regulate the amount of light that reaches the retina 22. The iris 34 divides the space between the cornea 14 and a lens 42 into an anterior chamber 46 and posterior chamber 50. The retina 22, which consists of nerve elements, can be considered a further internal covering disposed over the choroid 26. The nerve elements form the true receptive, light sensing portion for capturing visual impressions.

The retina 22 can be thought of as an outgrowth from the fore-brain, with the optic nerve 54 being a fiber tract connecting the retina with the fore-brain. A layer of special visual cells or photoreceptors called rods and cones lie just beneath a pigmented epithelium on the anterior wall of the retina 22. These cells transform physical energy in the form of light into nerve impulses transmitted along the optic nerve 54.

A vitreous body 58 resides between the lens 42 and the retina 22. The vitreous body 58 is a transparent gelatinous mass which fills the posterior four-fifths of the eye 10. The vitreous body 58 fills the space between the ciliary body 30 and the retina 22. A frontal saucer-shaped depression in the vitreous body 58 abuts a posterior portion of the lens 42. The lens 42 of the eye 10 is a transparent bi-convex body of crystalline appearance placed between the iris 34 and vitreous body 58. Its axial dimension varies with accommodation. It is the deformation of the lens 42 that enables the eye 10 to cause light rays from objects that are located at a range of distances from the eye to converge on the retina 22. A ciliary zonule 62, consisting of transparent fibers passing between the ciliary body 30 and the lens 42, holds the lens in position and enables the ciliary body 30 to act on the lens 42.

The cornea 14 is a fibrous portion of the eye 10 that highly light-transmissive. The curvature of the cornea 14 is somewhat greater than the rest of the eye 10 and is roughly spherical. Sometimes the cornea 14 is more curved in one meridian than another giving rise to astigmatism. Astigmatism, like myopia and hyperopia, discussed above, is a refractive error of the eye that can be treated by optic devices described herein. A central portion, e.g., a central approximate one-third, of the cornea is sometimes called the optical zone. Outward of the optic zone, the cornea 14 can include a slight flattening as the cornea thickens towards its periphery. Most of the refraction of the eye 10 takes place through the cornea 14.

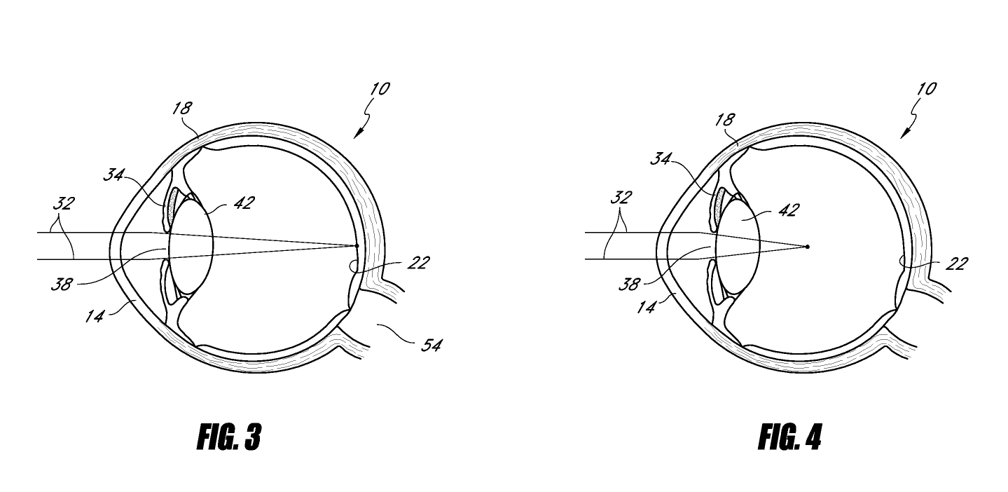

FIG. 2 shows a more detailed drawing of an anterior portion of the eye 10 that shows different layers of the cornea 14, including an outer layer called the epithelium 66 and an internal layer called the stroma 70. The epithelium 66 includes a thin layer of epithelial cells that act as a protective layer of the cornea 14. These epithelial cells are rich in glycogen, enzymes and acetylcholine and their activity regulates the corneal corpuscles and controls the transport of water and electrolytes through more posterior layers of the cornea 14, such as through lamellae of the stroma 70.

A Bowman's membrane 74 forms an anterior limiting lamina, positioned between the epithelium 66 and the stroma 70. The stroma 70 is comprised of lamella or layers having bands of fibrils parallel to each other and crossing the whole of the cornea 14. While most of the fibrous bands are parallel to the surface of the cornea 14, some are oblique, especially anteriorly. A membrane called the “Descemet's membrane” 78 forms a posterior limiting lamina and is a strong membrane sharply defined from the stroma 70.

The cornea 14 also includes posterior-most layer called the endothelium 82 that consists of a single layer of cells that aid in maintaining the transparency of the cornea. The eye 10 also includes a limbus 86 and conjunctiva 90. The limbus 86 is a transition zone between the conjunctiva 90 and sclera 18 and the cornea 14.

An ocular device, such as those disclosed herein, can be deployed in the cornea 14 using a variety of techniques and, as such, the terms “inlay” and “inlay lens” are sometimes used in connection with these ocular devices. For example, the ocular device disclosed herein may be implanted in the stromal layer 70 of the corneal 14 to provide refractive correction to the light passing through the cornea 14. Techniques that can be used for such placement include forming a corneal flap, forming a pocket in the cornea through a small surface cut, and placing any of these ocular devices in connection with another procedure that has created access to an internal layer of the cornea 14. Other ocular devices and corneal lenses that are suitable for compensating for refractive error or otherwise improving a patient's vision may be placed on the cornea, e.g., on or in the epithelium 66 of the eye, between the lens 42 and cornea 14, on or in the lens 42, attached to or on part of a phako lens, or in the anterior chamber 46 or posterior chamber 50.

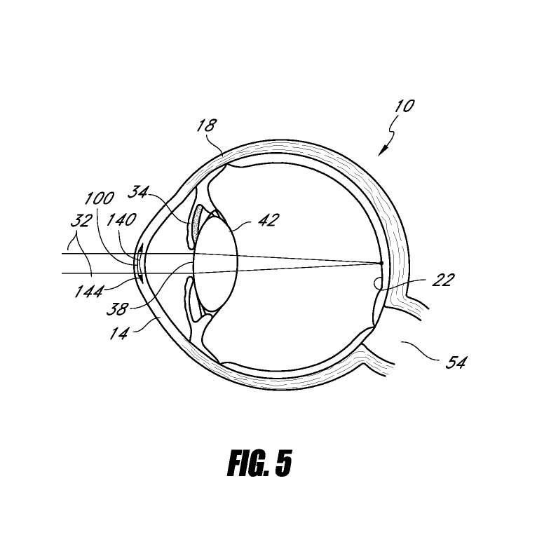

FIG. 3 shows an eye 10 having normal refractive capabilities. Essentially parallel light rays 32 as from a distant object that pass through the cornea 14 with the normal curvature are refracted by the cornea 14 and the lens 42 and converge near the retina 22 of the eye to produce an image. FIG. 4 illustrates, in contrast, an eye 10 that has a refractive defect or error. More particularly, the eye 10 is myopic. Here, light rays 32 that are parallel are refracted into focus within the vitreous body, at a point short of the retina when body structures that deform the lens 42 in accommodation are relaxed. The applicants have invented certain ocular devices can be implanted in the cornea to alter the refractive properties of the eye and to thereby compensate for the refractive error, such as those illustrated in FIG. 4.

FIG. 5 shows an ocular device 100 that is capable of compensating for refractive error of the eye 10 that can be implanted in the stromal layer 70 of the cornea 14 in a myopic eye. Here, the light rays 32 passing through the cornea 14 and through the ocular device 100 will be refracted at a smaller angle to compensate for the refractive error of the myopic eye and thus will converge at a more distant point, such as directly on the retina 22.

In addition to being able to compensate for refractive errors, the ocular device 100 can be configured with other advantageous features. For example, in one arrangement, the ocular device 100 is configured to lessen glare and other aberrant visual effects around an edge thereof. In another arrangement, the ocular device 100 may be additionally configured to increase the depth of focus of the patient's eye, thereby increasing the depth of field, i.e. the range of distance along the optical axis in which an object can moved without the image appearing to lose sharpness.

### II. Other Ocular Devices for Compensating for Refractive Errors

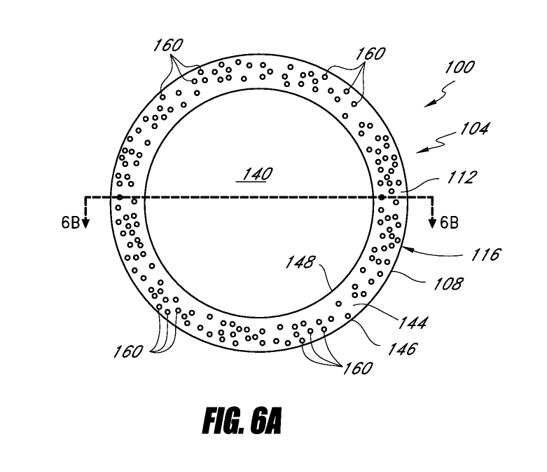

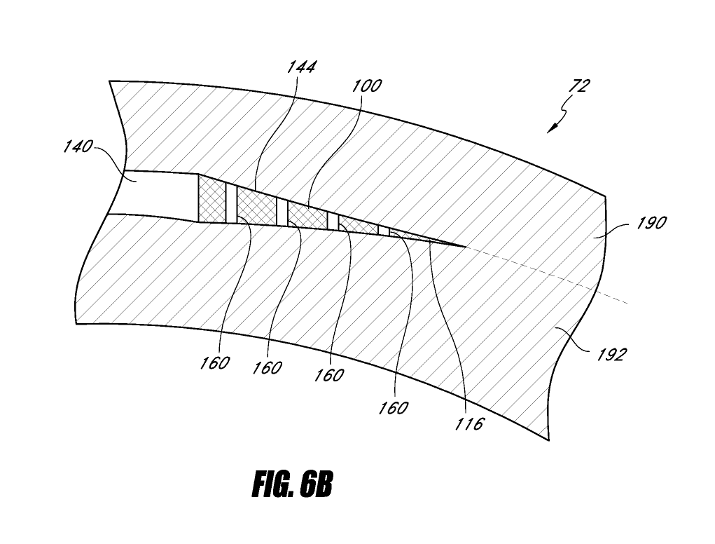

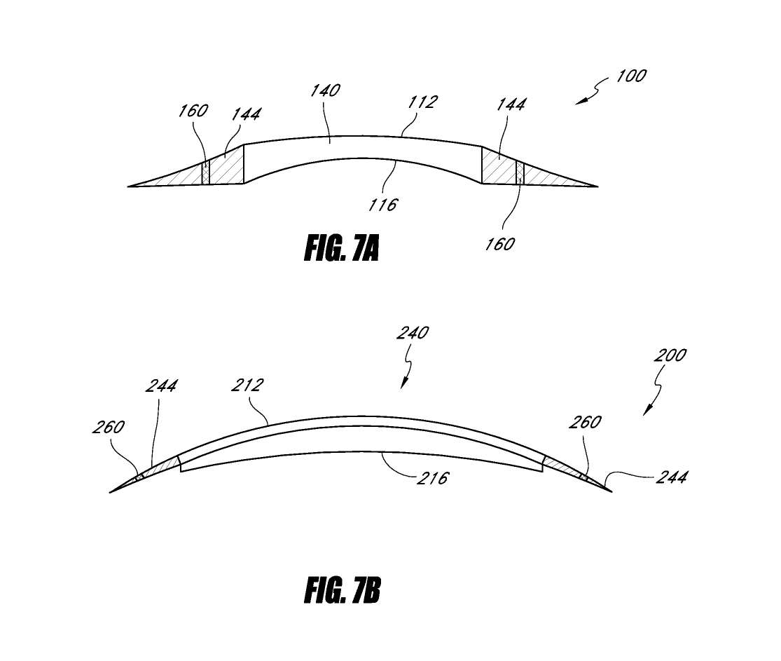

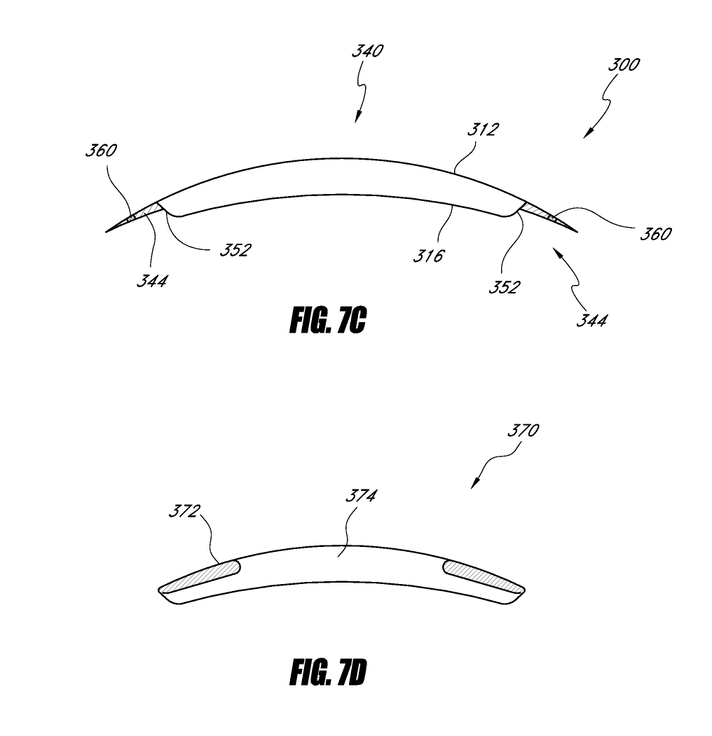

FIGS. 6A-7C illustrate further details of the ocular device 100 and variations thereof. The ocular device 100 can be configured as a lens that is suitable for deployment within the cornea, e.g., as a corneal lens. In one embodiment, the ocular device 100 is configured to be applied to the cornea of a patient, e.g., in a position between two layers of the cornea. A variety of techniques can be used to make the ocular device 100 suitable for positioning within the cornea, such as selecting a suitable thickness or range of thicknesses from anterior to posterior, selecting a material that is particularly compatible with corneal tissue, or selecting a suitable curvature. These features are discussed further below.

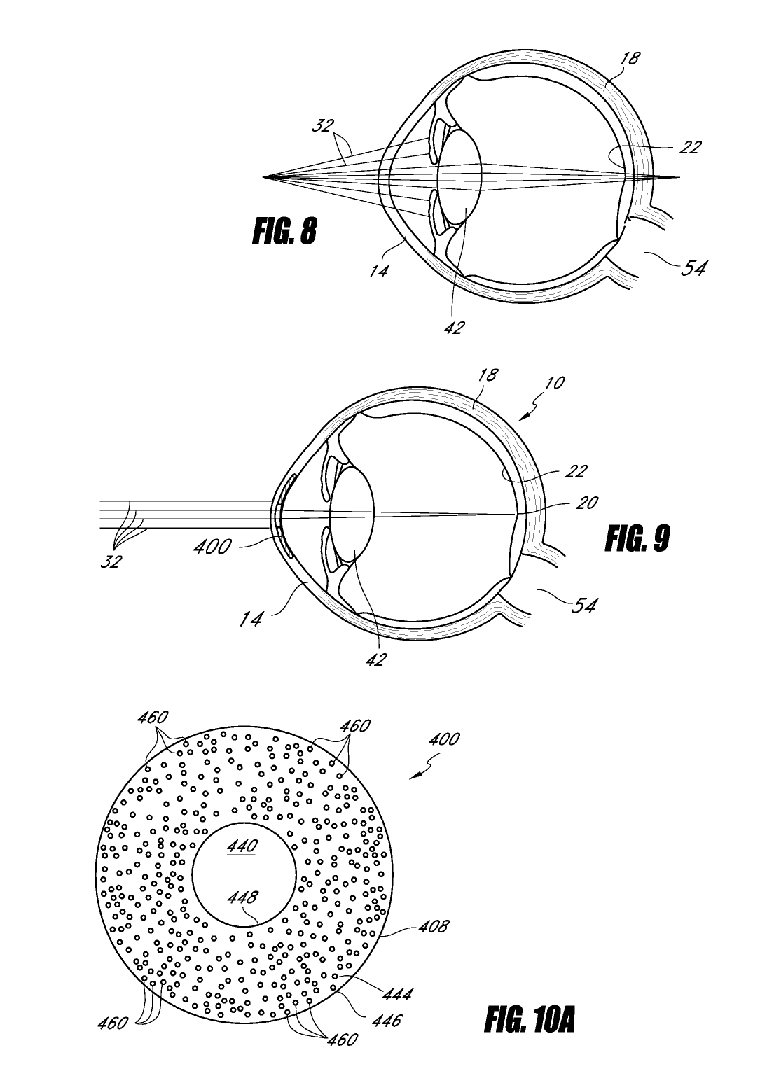

Preferably the ocular device 100 is capable of refracting light to compensate for a refractive error of the eye, as discussed further below. Some embodiments of the ocular device 100 include materials that provide a suitable refractive index to compensate for refractive error. Other embodiments rely on curvature of one or more surfaces of the ocular device to compensate for refractive error. As discussed in connection with FIGS. 8-10C, other embodiments may additionally rely on a pinhole or stenopaeic aperture to provide suitable compensation for loss of accommodation. Some embodiments use one or more of a suitable material, suitable curvature of at least one surface, a pinhole or stenopaeic aperture and other optical effects to compensate for refractive error and/or loss of accommodation, as discussed further below.

As shown in FIG. 6A , the ocular device 100 can include a lens body 104 having an outer perimeter 108 and an anterior surface 112 that extends to the outer perimeter 108. The lens body 104 also has a posterior surface 116 that extends to the outer perimeter 108. As discussed further below, the anterior and posterior surfaces 112, 116 can be configured to abut adjacent corneal layers when implanted. Preferably the ocular device 100 and particularly the anterior and posterior surfaces 112, 116 are configured to compatibly reside between such adjacent corneal layers. The outer perimeter 108 can take any suitable form. For example, the outer perimeter 108 can be generally circular, being defined by an outer circumference of the ocular device 100.

In certain embodiments, the lens body 104 includes a transmissive zone, or region, 140 and nontransmissive zone, or region, 144. The nontransmissive zone 144, where included, can be opaque in some embodiments. The transmissive zone 140 can be positioned at least partially in the optical zone of the cornea such that light entering the cornea and passing to the retina passes through the anterior and posterior surfaces 112, 116. In certain embodiments, the transmissive zone 140 can be substantially centered on or intersected by an optical axis of the eye, such as the line of sight and an axis passing through the center of the entrance pupil and the center of the patient's eyeball. The transmissive zone 140 is further configured to transmit at least a majority of the light that impinges thereon. In one embodiment, the transmissive region 140 transmits all or nearly all of the light in the visible range that impinges on the anterior surface 112. For example, in one embodiment, the transmissive zone 140 transmits at least about ninety percent, alternatively at least about eighty-five percent of the visible light incident on the anterior surface 112. In some cases, the transmissive zone 140 is configured to transmit at least about eighty percent of the visible light incident on the anterior surface 112. In some embodiments, the transmissive zone 140 can be considered a transparent region.

The transmissive zone 140 can be located at least partially within the outer region 108, as shown in FIG. 6A. In one embodiment, the transmissive zone 140 is completely surrounded by the outer region 108 of the ocular device 100. In some embodiments, the transmissive zone 140 is advantageously centrally located within the outer region 108 of the ocular device 100. In one embodiment, the geometric center of the transmissive zone 140 and the geometric center of the outer region coincide, e.g., at a central optic axis of the ocular device 100.

The transmissive zone 140 is large enough to cover a substantial portion of the optical zone of the cornea in one embodiment. For example, the transmissive zone 140 can cover more than half of the optical zone when the iris is fully dilated in one embodiment. In another embodiment, the transmissive zone 140 covers substantially the entire optical zone when the iris is fully dilated. In another embodiment, the transmissive zone 140 covers the entire optical zone when the iris is fully dilated. In one embodiment, the transmissive zone 140 covers more than half of the optical zone when the iris is fully constricted. In another embodiment, the transmissive zone 140 covers substantially the entire optical zone when the iris is fully constricted. In another embodiment, the transmissive zone 140 covers the entire optical zone when the iris is fully constricted. Other embodiments exploit a relatively small transmissive zone 140 to enhance transportation of nutrients between corneal tissues located anterior and posterior of the transmissive zone. Such small lens embodiments might permit the eye to operate around the transmissive zone 140 to provide multiple focalities.

The transmissive zone 140 can be formed with a suitable transverse dimension, e.g., a diameter, in the range of about 2.5 to about 3.0 mm, in one embodiment. The transmissive zone 140 can have a transverse dimension of at least about 2.5 mm. The transmissive zone 140 can be circular with a diameter of at least about 2.5 mm. For variation of the ocular devices 100 that have a transmissive zone 140 with a transverse dimension larger than 3.0 mm, more biocompatible materials or nutrient flow sustaining arrangements can be used to minimize nutrient depletion. Other materials can be used with variations of the ocular devices 100 that have smaller transmissive zone, e.g., that have diameters less than 2.5 mm.

In certain embodiments, the ocular device 100 can be configured such that a transverse dimension of the transmissive zone 140 is greater than that which would produce a pinhole effect. By making the transmissive zone 140 larger than that which would produce a pinhole effect, more light is permitted to reach the retina. Accordingly, the patient has a sense of greater illumination, especially during darker conditions such as while driving at night.

As discussed above, the transmissive zone 140 can be configured to alter the refractive properties of the eye to compensate for a refractive error of the eye in some embodiments. The refractive properties can be altered in one or more of a plurality of ways, for example, by providing a refractive power in the transmissive zone, by modifying the curvature of the cornea, or by providing a refractive power and by modifying curvature. In certain embodiments, the transmissive zone 140 may include a material with an index of refraction that lessens or steepens the angle of light passing therethrough. Such a lens could be configured with substantially the same curvature as that of a corresponding layer of the cornea, e.g., a layer that is adjacent to the transmissive zone 140 or to the anterior surface or posterior surface 112, 116. In some cases, the transmissive zone 140 can be made of such a material with a suitable curvature and with a thickness that does not disrupt the natural shape of the anterior surface of the cornea. In alternative embodiments, the anterior or posterior surfaces 112, 116 can be configured with an appropriate curvature to lessen or steepen the angle of light passing therethrough. In some embodiments, at least one of material selection and curvature of the anterior or posterior surface may be provided to steepen or lessen the angle of light passing through the ocular device 100.

FIGS. 7A-7C show an embodiment of a ocular device with a transmissive zone 140 that comprises a central lens portion having a refractive index substantially different from the index of refraction of the corneal tissue. The refractive index contributes to the refractive power of the transmissive zone 140, along with the geometry thereof. The refractive power of the lens may be selected to compensate for the mismatch between the refractive power of the eye and the length of the eye and thereby cause the transmitted light rays to properly converge on the retina. For example, the curvature of at least one of the anterior surface and posterior surface of the lens can be selected to augment and/or to provide a refractive power for correcting the refractive error of the eye. Different embodiments that compensate for refractive error in different manners are discussed below in connection with FIGS. 7A-7C.

For correcting myopia, or nearsightedness, a negative power, or diverging, lens can be used to spread the light passing through the lens. Such a negative power lens can cause the light rays passing through the transmissive zone to be refracted at a smaller angle, or spread, and therefore converge at a more distant point in the eye, such as directly on the retina. In certain embodiments, a negative power lens can be formed as a biconcave lens. A negative power meniscus lens also can be provided in which the relative curvatures of the anterior and posterior sides of the lens cause divergence or spreading of the light rays compared to the uncorrected eye. For example, as shown in FIG. 7A, the posterior surface 116 has a concave configuration with a curvature greater than that of the convex configuration of anterior surface 112. In this arrangement, the transmissive zone 140 is thicker at the periphery than near the center. This provides a negative power to the lens.

A positive power, or converging, lens, can be used to correct for hyperopia (farsightedness). Such a positive power lens causes the light rays passing through the transmissive zone 140 to be refracted at a greater angle, such that they converge at a nearer point in the eye than they would in the uncorrected eye. This preferably causes the rays to converge directly on the retina. A positive power lens may be formed as a biconvex lens, a plano-convex lens, or alternatively a positive power meniscus lens. For example, as shown in FIG. 7B, the posterior surface 216 and the anterior surface 212 of the lens portion can each have a convex configuration to provide a positive power to the lens and thereby correct for hyperopia. Alternatively, as shown in FIG. 7C, a positive power meniscus lens may be used to correct for hyperopia. Here, the anterior surface 316 may have a convex configuration with a greater curvature than that of the concave posterior surface 312. As such, the transmissive zone 340 is thicker in the center than at the periphery and thus provides a positive, or converging, power.

With reference to FIG. 6A, a substantially nontransmissive region 144 may extend between the outer perimeter 108 and the lens, or transmissive zone 140. The nontransmissive region 144 is generally located toward the periphery of the ocular device 100. In one embodiment, the nontransmissive region 144 includes an outer periphery 146 and an inner periphery 148. In the illustrated embodiment, a relatively sharp demarcation is provided between an outer region of the transmissive zone 140 and the inner periphery 146 of the nontransmissive region 144. In some embodiments, a more gradual transition can be provided between the transmissive zone 140 and the nontransmissive region 144. For example, various apodization techniques can be applied between the transmissive region 140 and the opaque region 144. One apodization technique that can be used is to gradually change the amount of transmission in a region between the transmissive and nontransmissive zones 140, 144. Another apodization technique that can be used is to provide an abrupt change in transmission between the transmissive and nontransmissive zones 140, 144 but vary the distance of this edge from a central portion of the zone 140, e.g., by making the boundary undulating or wavy. A variety of other apodization techniques are set forth in U.S. Pat. Nos. 5,662,706; 5,905,561; and 5,965,330, which are all hereby incorporated by reference herein in their entireties.

The outer periphery 146 of the nontransmissive region 144 may coincide with the outer perimeter 108 of the ocular device 100 in some embodiments. Alternatively, the outer periphery 146 may be contained within the outer perimeter 108 of the ocular device 100. The nontransmissive region 144 preferably extends between the outer perimeter 108 and the transmissive zone 140. The nontransmissive region 144 preferably is configured to block or substantially prevent transmission of a substantial portion of visible light incident on an anterior surface thereof. In one embodiment, the nontransmissive region 144 blocks more than half of the visible light incident on an anterior surface thereof. In an alternative embodiment, the nontransmissive region 144 blocks at least about sixty percent of the visible light incident on an anterior surface thereof. In an alternative embodiment, the nontransmissive region 144 blocks at least about seventy percent of the visible light incident on an anterior surface thereof. In an alternative embodiment, the nontransmissive region 144 blocks at least about eighty percent of the visible light incident on an anterior surface thereof. In another embodiment, the nontransmissive region 144 blocks ninety percent or more of the visible light incident on an anterior surface thereof. In an alternative embodiment, the nontransmissive region 144 is an opaque region that transmits no more than twenty percent of the visible light incident thereon. In certain embodiments, the nontransmissive region may be considered opaque.

In some embodiments, the nontransmissive region 144 provides an advantage of preventing distracting visual effects from being visible to the patient. For example, the nontransmissive region 144 can be configured to block enough light to eliminate distracting visual effects at the edge of the ocular device 100. The nontransmissive region 144 also can reduce glare and other distracting visual effects at the boundary between the ocular device 100 and adjacent corneal tissue, particularly the tissue that resides adjacent to the outer perimeter 108. Glare can occur due to the difference in refraction of the light that passes through the ocular device 100 and the light that passes through the adjacent corneal tissue and not through the ocular device 100. Such refractive difference can be significant enough to be noticed by a patient, and thus can be distracting. Accordingly, in some embodiments, such glare can be reduced by making the width of the nontransmissive region 144 large enough to sufficiently space the light passing through the transmissive zone 140 from the light passing through the cornea outside of the ocular device 100. By providing sufficient space between light passing through the transmissive zone 140 and the light passing through the cornea outside of the ocular device 100, the visibility of glare and other distracting visual effects due to the implantation of the ocular device 100 can be lessened or eliminated.

The nontransmissive region 144 preferably is configured in some embodiments to reduce a noticeable difference in refraction of light passing through the ocular device 100 in the optical zone of the cornea and light that passes through the optical zone around the device, e.g., outside the outer perimeter 108. As such, the nontransmissive region 144 may be arranged around the transmissive zone 140. In one embodiment, the nontransmissive region 144 completely surrounds the transmissive zone 140, forming an opaque, annular region surrounding the transmissive zone 140. The nontransmissive region 144 may have a transverse dimension that includes the width of the annulus. Where the nontransmissive region 144 completely surrounds the transmissive region 140, the nontransmissive region 144 may have a transverse dimension that is approximately two times the width of the annulus. In one embodiment, the nontransmissive region 144 comprises a circular annulus in which at least one perimeter thereof is substantially circular. In some embodiments, the circular annulus has an inner and an outer perimeter at least one of which is circular. A circular annulus could also have a wavy boundary that varies in distance from a central portion of the device 100 by an average amount around the boundary that lies on a circle. In some embodiments, the inner perimeter may abut the outer perimeter of the central transmissive zone 140.

In one embodiment, the inner perimeter of the annulus of the nontransmissive region 144 is circular, having a diameter of at least about 2.5 mm. In another embodiment, the inner perimeter of the annulus of the nontransmissive region 144 is circular, having a diameter of at least about 3.0 mm. In one embodiment, the combined width of the two portions of the nontransmissive region 144 on opposite sides of the transmissive region 140 is about 1.5, mm or more. In another embodiment, the combined width of the two portions of the nontransmissive region 144 on opposite sides of the transmissive region 140 is about 1.3 mm or more. In another embodiment, the combined width of the two portions of the nontransmissive region 144 on opposite sides of the transmissive region 140 is at least about 0.8 mm or more. In some embodiments, a transverse dimension of the transmissive zone 140 is greater than a transverse dimension of the nontransmissive region 144. The ocular device 100 preferably is configured such that an inner periphery of the nontransmissive region 144 has a transverse dimension that is greater than that which would produce a pinhole effect. Such an arrangement provides greater illumination, as discussed above, particularly in dark conditions. This configuration is particularly advantageous for patients that do not have problems with accommodation.

In an alternative embodiment, the nontransmissive region may have a transverse dimension sufficient to extend to a projection of the pupil of the eye. For example, the width of the nontransmissive region 144 extending across the transmissive region 140 can be about 8 mm or more. Here, the nontransmissive region 144 can substantially reduce glare by preventing light from being transmitted through adjacent corneal tissue. In such embodiments, the nontransmissive region 144 can be color matched to the patient's iris to minimize the visibility of the mask within the patient's eye.

Although in certain embodiments the nontransmissive region 144 is a peripheral region and is described as an “opaque” region, any construction that substantially prevents light from passing through the region 144 could provided at least some of the advantages described herein,- such as reducing glare or other distracting visual effect caused by the ocular device 100. Other optical phenomenon that can be provided in nontransmissive region 144 to prevent transmission therein are described in U.S. Patent No. 6,554,424, issued April 29, 2003, which is hereby incorporated by reference herein in its entirety. Such phenomena can include one or more of reflection of light in the nontransmissive region 144, diffraction of light in the nontransmissive region 144, and scattering of light in the nontransmissive region 144, alone or in combination with light absorption to provide at least one of the advantages described herein.

Where the nontransmissive region 144 is configured to be opaque, the opacity can be provided by forming the region 144 of an opaque material. In another embodiment, opacity can be provided by forming the opaque region 144 of a light absorbing material that is embedded in another material that can be clear or opaque. For example, the opaque region 144 can be formed by mixing together a suitable polymer material and sufficient quantity of an opacification agent to provide adequate absorption of light and prevent a refractive difference across the transition from the transmissive zone to the opaque region that would be noticeable to the patient. Carbon is one example of a suitable opacification agent. In one embodiment, the carbon can include carbon black and/or small, e.g., submicron, powdered carbon particles.

In some embodiments, the ocular device 100, particularly the nontrasmissive region 144, has a very high surface to volume ratio and is exposed to a great deal of sunlight following implantation, the mask preferably comprises a material which has good resistance to degradation, including from exposure to ultraviolet (UV) or other wavelengths of light. Polymers including a UV absorbing component, including those comprising UV absorbing additives or made with UV absorbing monomers (including co-monomers), may be used in forming masks as disclosed herein which are resistant to degradation by UV radiation. Examples of such polymers include, but are not limited to, those described in U.S. Pat. Nos. 4,985,559 and 4,528,311 and U.S. application Ser. No. 11/404,048, the disclosures of which are hereby incorporated by reference in their entireties. In a preferred embodiment, the mask comprises a material which itself is resistant to degradation by UV radiation. In one embodiment, the mask comprises a polymeric material which is substantially reflective of or transparent to UV radiation.

Alternatively, the ocular device 100 may include a component which imparts a degradation resistive effect, or may be provided with a coating, preferably at least on the anterior surface, which imparts degradation resistance. Such components may be included, for example, by blending one or more degradation resistant polymers with one or more other polymers. Such blends may also comprise additives which provide desirable properties, such as UV absorbing materials. In one embodiment, blends preferably comprise a total of about 1-20 wt. %, including about 1-10 wt. %, 5-15 wt. %, and 10-20 wt. % of one or more degradation resistant polymers. In another embodiment, blends preferably comprise a total of about 80-100 wt. %, including about 80-90 wt. %, 85-95 wt. %, and 90-100 wt. % of one or more degradation resistant polymers. In another embodiment, the blend has more equivalent proportions of materials, comprising a total of about 40-60 wt. %, including about 50-60 wt. %, and 40-50 wt. % of one or more degradation resistant polymers. Ocular devices disclosed herein may also include blends of different types of degradation resistant polymers, including those blends comprising one or more generally UV transparent or reflective polymers with one or more polymers incorporating UV absorption additives or monomers. These blends include those having a total of about 1-20 wt. %, including about 1-10 wt. %, 5-15 wt. %, and 10-20 wt. % of one or more generally UV transparent polymers, a total of about 80-100 wt. %, including about 80-90 wt. %, 85-95 wt. %, and 90-100 wt. % of one or more generally UV transparent polymers, and a total of about 40-60 wt. %, including about 50-60 wt. %, and 40-50 wt. % of one or more generally UV transparent polymers. The polymer or polymer blend may be mixed with other materials as discussed below, including, but not limited to, opacification agents, polyanionic compounds and/or wound healing modulator compounds. When mixed with these other materials, the amount of polymer or polymer blend in the material which makes up the mask is preferably about 50%-99% by weight, including about 60%-90% by weight, about 65-85% by weight, about 70-80% by weight, and about 90-99% by weight.

In general, the nontransmissive region 144 can include an opacification agent to prevent transmission of at least some light, e.g., visible light. Some opacification agents, such pigments, which are added to blacken, darken or opacify portions of the ocular device 100 (or the other ocular devices disclosed herein) may cause the ocular device to absorb incident radiation to a greater degree than materials not including such agents. To enhance the resistance to UV degradation, the ocular device 100 can be made at least in part of a material which is itself resistant to degradation such as from UV radiation, or that is generally transparent to or non-absorbing of UV radiation. One class of materials that can be used includes highly fluorinated polymers, including those in which the number of carbon-fluorine bonds in the polymer equals or exceeds the number of carbon-hydrogen bonds in the polymer. PVDF is one highly fluorinated polymer that could be used advantageously in an ocular device disclosed herein. Use of a highly fluorinated polymer, such as PVDF, or another highly UV resistant and degradation resistant material which is highly transparent to UV radiation, allows for greater flexibility in the selection of the opacification agent because possible damage to the polymer caused by selection of a particular opacification agent is greatly reduced. More details concerning the use of highly fluorinated polymers, such as PVDF, alone or in combination with carbon black or other suitable opacification agents and other additives that provide advantageous features, such as polyanionic compounds like proteoglycans and glycosaminoglycans, can also be incorporated into an ocular device disclosed herein. Additional polyanionic compounds and other useful additives include glucose-6 phosphate, dermatan sulfate, chondroitin sulfate, keratan sulfate, heparan sulfate, heparin, dextran sulfate, hyaluronic acid, pentosan polysulfate, xanthan, carrageenan, fibronectin, laminin, chondronectin, vitronectin, poly L-lysine salts, and alginate. In some embodiments, a useful additive includes dextran sulfate.

In addition, it may be useful to incorporate into the ocular device 100 (or another ocular device disclosed herein) a wound healing modulator, which can be loaded into the polymeric material and/or bound to at least one of the anterior surface and the posterior surface. The wound healing modulator can be a compound selected from the group consisting of antibiotics, antineoplastics, antimitotics, antimetabolics, anti-inflammatories, immunosupressants, and antifungals. In certain embodiments, the wound healing modulator compound can be selected from the group consisting of fluorouracil, mitomycin C, paclitaxel, ibuprofen, naproxen, flurbiprofen, carprofen, suprofen, ketoprofen, and cyclosporins.

Preferred degradation resistant polymers that can be used in the ocular devices disclosed herein include halogenated polymers. Preferred halogenated polymers include fluorinated polymers, that is, polymers having at least one carbon-fluorine bond, including highly fluorinated polymers. The term “highly fluorinated” as it is used herein, is a broad term used in its ordinary sense, and includes polymers having at least one carbon-fluorine bond (C—F bond) where the number of C—F bonds equals or exceeds the number of carbon-hydrogen bonds (C—H bonds). Highly fluorinated materials also include perfluorinated or fully fluorinated materials, materials which include other halogen substituents such as chlorine, and materials which include oxygen- or nitrogen-containing functional groups. For polymeric materials, the number of bonds may be counted by referring to the monomer(s) or repeating units which form the polymer, and in the case of a copolymer, by the relative amounts of each monomer (on a molar basis).

Preferred highly fluorinated polymers include, but are not limited to, polytetrafluoroethylene (PFTE or Teflon®), polyvinylidene fluoride (PVDF or Kynar), poly-1,1,2-trifluoroethylene, and perfluoroalkoxyethylene (PFA). Other highly fluorinated polymers include, but are not limited to, homopolymers and copolymers including one or more of the following monomer units: tetrafluoroethylene —(CF₂—CF₂)—; vinylidene fluoride —(CF₂—CH₂)—; 1,1,2-trifluoroethylene —(CF₂—CHF)—; hexafluoropropene —(CF(CF₃)—CF₂)—; vinyl fluoride —(CH₂—CHF)— (homopolymer is not “highly fluorinated”); oxygen-containing monomers such as —(O—CF₂)—, —(O—CF₂—CF₂)—, —(O—CF(CF₃)—CF₂)—; chlorine-containing monomers such as —(CF₂—CFCl)—. Other fluorinated polymers, such as fluorinated polyimide and fluorinated acrylates, having sufficient degrees of fluorination are also contemplated as highly fluorinated polymers for use in ocular devices disclosed herein according to preferred embodiments. The homopolymers and copolymers described herein are available commercially and/or methods for their preparation from commercially available materials are widely published and known to those in the polymer arts.

Although highly fluorinated polymers are preferred, polymers having one or more carbon-fluorine bonds but not falling within the definition of “highly fluorinated” polymers as discussed above, may also be used. Such polymers include co-polymers formed from one or more of the monomers in the preceding paragraph with ethylene, vinyl fluoride or other monomer to form a polymeric material having a greater number of C—H bonds than C—F bonds. Other fluorinated polymers, such as fluorinated polyimide, may also be used. Other materials that could be used in some applications, alone or in combination with a fluorinated or a highly fluorinated polymer, are described in U.S. Pat. No. 4,985,559, U.S. Pat. No. 4,538,311, and U.S. application Ser. No. 11/404,048, all of which are hereby incorporated by reference herein in their entirety.

The preceding definition of highly fluorinated is best illustrated by means of a few examples. One preferred UV-resistant polymeric material is polyvinylidene fluoride (PVDF), having a structure represented by the formula: —(CF₂—CH₂)ₙ—. Each repeating unit has two C—H bonds, and two C—F bonds. Because the number of C—F bonds equals or exceeds the number of C—H bonds, PVDF homopolymer is a “highly fluorinated” polymer. Another material is a tetrafluoroethylene/vinyl fluoride copolymer formed from these two monomers in a 2:1 molar ratio. Regardless of whether the copolymer formed is block, random or any other arrangement, from the 2:1 tetrafluoroethylene:vinyl fluoride composition one can presume a “repeating unit” comprising two tetrafluoroethylene units, each having four C—F bonds, and one vinyl fluoride unit having three C—H bonds and one C—F bond. The total bonds for two tetrafluoroethylenes and one vinyl fluoride are nine C—F bonds, and three C—H bonds. Because the number of C—F bonds equals or exceeds the number of C—H bonds, this copolymer is considered highly fluorinated.

Certain highly fluorinated polymers, such as PVDF, have one or more desirable characteristics, such as being relatively chemically inert and having a relatively high UV transparency as compared to their non-fluorinated or less highly fluorinated counterpart polymers. Although the applicant does not intend to be bound by theory, it is postulated that the electronegativity of fluorine may be responsible for many of the desirable properties of the materials having relatively large numbers of C—F bonds.

In certain embodiments wherein the opaque region extends to the edges of the patient's iris, a color pigment may be mixed with a partially fluorinated polymer to provide opacity. Alternatively, the color pigment and carbon black particles may both be used to provide opacity to the partially fluorinated polymer. Further details of materials and additives that can be used in the ocular devices disclosed hererin, e.g., in the nontransmissive region 144, are discussed in U.S. patent application Ser. No. 11/404,048, filed Apr. 13, 2006, which is hereby incorporated by reference herein in its entirety.

The ocular device 100 also is configured in some embodiments to enhance the ability of the device to maintain its position relative to a feature of the eye, such as the line of sight of the eye or a constricted or dilated pupil. For some embodiments, the performance of the ocular device 100 is enhanced by enabling the device to maintain its position relative to the line of sight. In certain embodiments, such as correcting an astigmatism, wherein the transparent region has zones of different refractive power for correcting the astigmatism, the ability of the ocular device 100 to maintain a selected position, e.g., a selected angular position, is important. In the example of astigmatism, ability of the ocular device 100 to hold its position enables a particular zone of the transparent region 140 to be aligned as prescribed, thus enabling the device 100 to compensate for a first deficiency in a first region of the cornea and to compensate for a second deficiency in a second region of the cornea. Accordingly, the ocular device 100 can be used to provide more precise correction of refractive errors in the eye than were the ability to hold position not present.

FIGS. 6A-6B show that in one embodiment, the ocular device 100 is configured to engage tissue of the cornea to reduce the tendency of the ocular device to move within the cornea after it has been implanted. Such engagement preferably does not include tissue in-growth, which could affect the removability of the device or cause aberrant visual effects. Movement of the device 100 is possible because the cornea is a highly layered structure, as discussed above. When two naturally adjacent layers are separated, adjacent space around an implant positioned therein may be created or pressure applied to the eye such as by rubbing can cause an implant positioned therein to further separate the layers to permit movement of the device.

In one form, the ocular device 100 is configured to hold its position after being implanted by being provided with a plurality of recesses 160 that extend from at least one of the anterior and posterior surfaces 112, 116. The recesses 160 can take any suitable form. For example, the recesses 160 can extend from the anterior surface 112 to the posterior surface 116. The recesses 160 can be cylindrical channels, which can have a circular cross-section. Preferably, the recesses 160 are provided toward the periphery of the ocular device 100, e.g., in the nontransmissive region 144. Depending on the thickness of the ocular device 100, the recesses 160 can be small holes dispersed about the device, e.g., in the nontransmissive region. In one embodiment, the recesses 160 are confined to the nontransmissive region 144. In one embodiment, the recesses 160 do not extend into the transmissive region 140. The recesses 160 can be located throughout a peripheral region, such as the nontransmissive region 144.

In the illustrated embodiment, the recesses 160 are provided throughout the nontransmissive region 144 and are confined thereto. Preferably, the recesses 160 are configured to not produce visible optical effects that would be distracting to the patient. Such optical effects are sometimes produced by locating the recesses at regular positions. Accordingly, the recesses 160 can be located at irregular positions to minimize visible optical effects, such as diffraction patterns. Some or all of the recesses 160 can be located at random positions to minimize visible optical effects, such as diffraction patterns. A variety of techniques for locating apertures that could be used to locate the recesses are discussed in U.S. Pat. No. 7,628,810, issued Dec. 8, 2009.

FIG. 6B illustrates that the recesses 160 can be configured to receive adjacent tissue, e.g., corneal tissue, to reduce the tendency of the ocular device 100 to move within the eye after being implanted. For example, once the ocular device 100 is implanted in the stromal layer 72 of the cornea, corneal tissue adjacent to the recesses 160 swells or expands into the recesses 160. By permitting corneal tissue to expand into the recesses 160, the likelihood of the ocular device 100 becoming displaced within the cornea after being implanted or to otherwise moving relative to the eye can be reduced. However, the nontransmissive zone 144 is configured to be relatively thin, and thus the recesses 160 also are relatively short. Because the recesses 160 are short, the surrounding corneal tissue only expands into the recesses. In some cases, the expansion of the corneal tissue into the recesses 160 is due to osmotic pressure or an effect similar to a capillary effect. One advantage of the embodiments discussed herein is that tissue is drawn into the recesses 160, it is believed that such drawn-in tissue completely fills the short recesses 160 and thus prevents fibrous ingrowth of new tissue. Thus, the ocular device 100 preferably remains removable without damage or scarring to the adjacent corneal tissue. Fibrous ingrowth is not preferred because it makes removal of the device more challenging. Nevertheless, in some cases the recesses 160 do permit some fibrous ingrowth, which does not affect the performance of the ocular device 100.

Because the recesses 160 can reduce movement of the ocular device 100, the recesses can be thought of as increasing the adhesion or grip of the device to the eye. As discussed above, the ability to maintain the position of a portion of the ocular device 100 relative to an ocular feature, such as the line of sight, can be important to the performance of the device. For example, in some embodiments, locating an optic axis of the ocular device 100 near or on the line of sight of the eye to which the device is applied can improve the performance of the device. As discussed further below, any suitable technique for aligning the optic axis of the ocular device with the line of sight can be used, including using a centration agent (such as light, pilocarpine, or another pharmacologic agent) to increase the correlation between a visible ocular feature and the line of sight, or more directly locating the line of sight, such as by having a patient align two targets that are at different distances from the patient. More details on locating positioning the ocular device 100 relative to an ocular feature are set forth in U.S. patent application Ser. No. 10/854,032, filed May 26, 2004 and entitled “METHOD AND APPARATUS FOR ALIGNING A MASK WITH THE VISUAL AXIS OF AN EYE,” in U.S. patent applications Ser. No. 11/257,505, filed Oct. 24, 2005, and entitled “SYSTEM AND METHOD FOR ALIGNING AN OPTIC WITH AN AXIS OF AN EYE,” both of which are hereby incorporated by reference in their entirety.

The configuration of the recesses 160 can be selected to provide an adequate amount of gripping or position holding capability. In one embodiment, the recesses 160 are so configured by making them large enough to admit a sufficient amount of tissue to prevent movement of the ocular device 100. The recesses 160 can have a transverse dimension of at least about 0.015 mm. In one embodiment, the recesses 160 are formed with a diameter of about 0.015 mm or more. In another embodiment, the recesses 160 have a diameter of about 0.020 mm. In another embodiment, the recesses 160 have a diameter of about 0.025 mm. In another embodiment, the recesses 160 have a diameter in the range of about 0.020 mm to about 0.029 mm. In a further embodiment, the recesses 160 have a diameter up to about 0.075 mm. In one embodiment, as discussed above, the recesses 160 are cylindrical, having a circular cross-section and having a diameter with any of the foregoing dimensions.

The recesses 160 preferably also are configured to maintain the transport of one or more nutrients across the device 100. Preferably, the recesses 160 provide sufficient flow of one or more nutrients across the device 100 to prevent depletion of nutrients in the first corneal layer 190 adjacent the anterior surface 112 of the device 100. One nutrient of particular importance to the viability of the adjacent corneal layers is glucose. The transportation of glucose across the corneal tissue may be affected by the depth the device is implanted in the cornea, the thickness of the device, the permeability of the device and the number and size of the nutrient holes (e.g. porosity) provided in the device. For example, in certain embodiments, the recesses 160 may be configured to provide sufficient flow of glucose across the device 100 between the corneal tissue layers adjacent the device 100 to prevent glucose depletion that would harm the adjacent corneal tissue.

In one embodiment, the recesses 160 are configured to prevent depletion of more than about 5 percent of glucose (or other biological substances) in tissue of at least one of the first corneal layer 190 and the second corneal layer 192 adjacent to the nontransmissive region 144. In another embodiment, the recesses are configured to prevent glucose depletion of more than about 32% of glucose (or other biological substances) in tissue of at least one of the first corneal layer 190 and the second corneal layer 192 across the width of the device 100. Thus, the device 100 is capable of substantially maintaining nutrient flow (e.g., glucose flow) between adjacent corneal layers.

The recesses 160 can be located in particular regions of, e.g., in any of four quadrants of, the ocular device 100. Alternatively, the recesses 160 can be located in a smaller region of the ocular device 100. FIG. 6A shows the recesses 160 dispersed throughout the nontransmissive region 144. Preferably the recesses 160 are located at irregular positions, or are otherwise irregularly formed to reduce or substantially prevent the recesses from producing distracting optical effects. For example, in certain embodiments, the recess pattern or spacing may be non-uniform, e.g., random, the recesses may be non-uniform in shape and/or the recesses may be non-uniform in orientation. In alternative embodiments, the random pattern may be modified to enhance a performance characteristic of the device. More details on non-uniform and variations on random spacing and configuration of the recesses 160 can be found in U.S. patent application Ser. No. 11/417,895, filed May 3, 2006 and entitled “OPTICAL MASK FOR IMPROVING THE DEPTH OF FOCUS AND METHODS FOR IMPROVING DEPTH OF FOCUS,” hereby incorporated by reference in its entirety.

The ocular device 100 preferably is suitable for implantation between layers of the cornea 14 of an eye 10. In one embodiment, the posterior surface 116 is configured to reside adjacent a corneal layer. In one embodiment the anterior surface 112 also is configured to reside adjacent a corneal layer. In one arrangement, the anterior surface 112 is configured to reside adjacent a first corneal layer 190 and the posterior surface 116 is configured to reside adjacent a second corneal layer 192. Where the ocular device 100 is to be implanted in the cornea, the first and second corneal layers 190 and 192 may be discrete layers of the cornea, e.g., adjacent layers within the stroma, or any of the other layers discussed herein. As discussed above, in certain embodiments, the ocular device 100 may be implanted at a sufficient depth to reduce glucose depletion. For example, in certain embodiments, the device 100 is preferably implanted at a depth of between about 300-400 microns within the corneal tissue to minimize the glucose (or other nutrient) depletion to the anterior layers of the cornea. Implantation at other depths is also possible, as discussed below.

In one embodiment, the ocular device 100 has a thickness that enables the device to reside within the cornea. For example, the ocular device 100 can have a thickness that enables the device to reside between adjacent layers without requiring a separate method step of removing corneal layers to accommodate the device. In some embodiments, the ocular device 100 has a thickness within the transmissive region of less than about 0.4 mm. In certain embodiments, a constant thickness for the central transmissive region 140 can be used if the region 140 is otherwise configured to provide refractive correction or power, e.g., by being formed of a material with a selected refractive index. Alternatively, a non-constant thickness, as shown in FIGS. 7A-7C, may be easily adapted to treat a wide variety of patients. The non-constant thickness may result from the selection of surface profiles for the anterior and posterior surfaces of the ocular devices, e.g., the surfaces 112, 116, 212, 216, and 312, 316, for creating the positive and negative power lenses described above. In some embodiments, thicker devices can be accommodated by removing at least a portion of a corneal layer to accommodate the device. In certain embodiments, the non-constant thickness of the device may be configured to provide additional refractive correction by altering the curvature of the anterior or posterior surface of the cornea. Alternatively, the transmissive region can be made of a material having the same or substantially same refractive index as the cornea and thus the change in curvature due to the non-constant thickness of the device may provide the dominant contribution to the refractive correction. The nontransmissive region (or skirt) 144, 244, 344 can have a tapering thickness to minimize any gaps between the corneal tissue layers at the edges of the ocular device and thereby prevent tissue growth around the edges of the ocular device. For example, in one embodiment illustrated in FIG. 6B, the thickness of the ocular device gradually decreases from adjacent to the transmissive region to adjacent to the outer perimeter. This permits the ocular device to better conform with the adjacent corneal layers, preventing large gaps from forming at the edges of the device. By eliminating or reducing the size of such gaps, the formation of fibrous growths or other distracting results can be eliminated or reduced.

A variety of techniques can be used to make the ocular device 100 more suitable for implantation on or in the cornea. For example, the lens body 104 can include a biocompatible material. In the cornea, biocompatibility can be a function of the ability of a structure to maintain the biological integrity of adjacent structures. Maintaining biological integrity of adjacent structures can involve maintaining the flow of one or more nutrients such as glucose between two areas, e.g., between two adjacent layers, of the cornea. In one embodiment, the transmissive zone 140 does not have a plurality of recesses extending therethrough for providing nutrient transport, but is made of a high water content material, such as a hydrogel. Hydrogels comprise one class of materials that can be used for the transmissive zone 140. Alternatively, other similar materials that are able to transport nutrients, e.g., by having a high water content, can also be used. For example, in certain embodiments, materials having a water content of at least 25% and as much as 95% or more when immersed in normal saline at standard temperature and pressure (STP) can be used to construct the transmissive zone. In alternative embodiments, materials having a water content of at least 30% when immersed in normal saline at STP can be used to construct the transmissive zone 140. In alternative embodiments, materials having a water content of at least 35% when immersed in normal saline at STP can be used to construct the transmissive zone 140. In alternative embodiments, materials having a water content of no about 49% when immersed in normal saline at STP can be used to construct the transmissive zone. In alternative embodiments, materials having a water content of no more than 55% when immersed in normal saline at STP can be used to construct the transmissive zone.

In certain embodiments, the material may be further configured to expand by at least about 25% in volume when immersed in normal saline at equilibrium at STP. Such expansion can be used to couple transmissive and nontransmissive regions of an ocular device as discussed below.

The nontransmissive region 144 preferably comprises a plurality of holes for transportation of nutrients between the adjacent corneal layers and therefore does not require a material with a high water content. Accordingly, the nontransmissive region 144 can include a material having a water content of no more than 10% when immersed in normal saline at equilibrium at STP.

In certain embodiments, an ocular device 370 includes a hydrogel inlay with a nontransmissive region 372 and a transmissive region 374, as depicted in FIG. 7D. The nonransmissive region 372 can be an opaque region. In certain embodiments, the entire ocular device 370 includes a hydrogel. The nontransmissive region 372 can be configured as other nontransmissive regions described herein. For example, the nontransmissive region 372 can be a generally annular shaped structure, e.g. circular or any other suitable shape, that is disposed at least partially about the transmissive region 374. In certain embodiments, the nontransmissive region 372 is located adjacent to an outer perimeter of the ocular device 370.

The nontransmissive region 372 can also be located at a distance from the outer perimeter of the ocular device 370. In certain embodiments, the hydrogel inlay can be substantially unperforated, e.g., lacking in nutrient transport holes, because the hydrogel is able to transport nutrients without such structures. The nontransmissive region 372 can be formed using any suitable technique for opacifying the portion 372. One class of techniques that can apply to a hydrogel inlay is one or more processes similar to those used to form tattoos in skin. For example, an ink can be applied to, embedded in, or injected into the body of the device 370. In some embodiments, the nontransmissive region 372 can be printed onto the hydrogel inlay.

Other materials that have advantageous properties and that can be used for the transmissive region 140 include PMMA and polysulphones. Lower refractive index materials, such as PVDF, also could be used for a lens depending on the clarity and lens power required. Alternatively, in certain embodiments, a nutrient transport structure within the central transmissive zone may be provided by providing holes, similar to recesses 160 in the nontransmissive region 144, in the transmissive zone 140 as well. To prevent distortions in the transmission of light within the transmissive zone 140, the holes may be provided with features for preventing tissue ingrowth in the holes. For example, a hydrogel material or any other suitable material can be used to fill the recesses within the transmissive zone 140, thereby preventing tissue ingrowth while maintaining the light transmitting quality of the holes. Where the size of the transmissive region 140 is smaller, materials that are less able to transport nutrients can be used without compromising the biological integrity of the cornea. Materials that can be used in smaller devices are disclosed in U.S. Pat. No. 5,336,261, which is hereby incorporated by reference herein.

FIG. 7B depicts a cross-sectional view of another embodiment of an ocular device 200, which as discussed above may include a convex-convex construction. The ocular device 200 is similar to the ocular device 100, except as set forth below. Compatible structures of the ocular devices 100, 200 can be interchanged. The ocular device 200 has a transmissive region 240 and a nontransmissive portion 244. The nontransmissive portion 244 can have a skirt-like structure. As used herein, a “skirt-like” structure is a generally annular shaped structure, e.g. circular or any other suitable shape, that is disposed at least partially about the transmissive portion 240. As discussed further below, the skirt-like structure 244 can be an opaque or otherwise light blocking or nontransmissive extension of the transmissive portion 240. The nontransmissive portion 244 can be an extension of a separate structure located between the transmissive portion and the nontransmissive portion. The nontransmissive portion 244 can be located adjacent to an outer perimeter 208 of the ocular device 200. In certain embodiments, the nontransmissive portion 244 can be configured as a relatively thin skirt that is disposed about the transmissive portion 240. In one variation, the nontransmissive portion 244 does not have the same thickness as the transmissive portion 240. The nontransmissive portion 244 can be thinner than the transmissive portion 240, e.g., having a thickness of one-half of or less than one-half of the thickness of the transmissive portion 240. Where the nontransmissive skirt 244 is thinner than the transmissive portion 240, the nontransmissive skirt 244 can be coupled with an anterior surface 212 or a posterior surface 216 thereof. As discussed above, in certain embodiments, the thickness of the nontransmissive portion 244 decreases toward the periphery such that any gaps between the adjacent corneal tissue layers at the edges of the device are minimized. There are advantages to making the nontransmissive portion 244 thinner than the transmissive portion 240. For example, the nontransmissive portion need not have a geometry selected to provide a refractive effect. Instead, the nontransmissive portion can provide at least one of anchoring properties, aberrant visual effect depression properties, and nutrient transport properties. Depending upon the construction of the nontransimssive portion 244, one or more of these properties can be provided with a structure that can be thinner than the transmissive portion 240. By making the nontransmissive portion 244 thinner, the ocular device 200 can be better tolerated in the patient's cornea.

The ocular device 200 can be configured in any manner described above in connection with the ocular device 100 to be positioned within the cornea. At least one of the anterior and posterior surfaces 212, 216 of the ocular device 200 can be configured to reside adjacent to or to abut corneal tissue. In one variation, the anterior surface 212 has a curvature that is similar to the curvature of a first corneal layer. As discussed above, the anterior and posterior surfaces 212, 216 of the transmissive portion 240 can be configured to provide a positive power, e.g., by being convex in shape, which is conducive to compensating for hyperopia. Alternatively, the transmissive region 240 could be constructed of a material that provides a refractive index capable of improving a refractive error such as hyperopia, and thus could have other shapes as well.

As discussed above in connection with the ocular device 100, the ocular device 200 can have recesses 260 that are configured to enable the ocular device 200 to sufficiently grip adjacent corneal tissue such that the ocular device will not migrate in the eye after implantation. The recesses 260 can be similar to the recesses 160. Where the thickness of the opaque portion 244 is less than the thickness of the transmissive region 240, the length of the recesses 260 may be shorter than that of the recesses 160.

FIG. 7C is a cross-sectional view of another embodiment of an ocular device 300. The ocular device 300 is similar to the ocular devices 100, 200 except as set forth below. Compatible structures of the ocular device 300 and either of the ocular devices 100 and 200 can be interchanged. Also, compatible structures of the ocular devices 100, 200, 300 and any of the devices disclosed in any of the references incorporated by reference can be interchanged.

The ocular device 300 includes a transmissive portion 340 that is generally centrally located within the device. Disposed about the transmissive portion 340 is a nontransmissive portion 344. The nontransmissive portion 344 can be opaque or can be made nontransmissive in any other manner, such as by use of an optical effect, as discussed above: In one embodiment, the ocular device 300 has an anterior surface 312 and a posterior surface 316. In one embodiment, the anterior surface 312 is configured to reside adjacent to or abut corneal tissue, as discussed above. The posterior surface 316 preferably also is so configured. As discussed above, the curved posterior and anterior surfaces create a meniscus lens configuration in the transmissive portion 340 that is thicker in the middle than near the nontransmissive portion 344, thus providing a positive power. However, other suitable shapes, including negative meniscus lens, a biconvex lens or a planar convex lens could be used to provide the required refractive correction. In addition, cylindrical shapes could be used for astigmatism.

In one arrangement, the nontransmissive portion 344 includes a peripherally located region 346 that is similar to the nontransmissive region 244. The peripherally located region 346 preferably is opaque or nontransmissive. The nontransmissive portion 344 can be configured as an annular skirt-like structure that extends all the way around the transmissive region 340. The nontransmissive portion 344 can have a thickness that is less than the thickness of the transmissive region 340. By making the nontransmissive portion 344 thinner than the transmissive portion 340, the ocular device 300 can be better tolerated within the eye of the patient. The nontransmissive portion 344 can be coupled with the transmissive region 340 adjacent to at least one of the anterior and posterior surfaces 312, 316 or can be coupled thereto at a location mid-way between the anterior and posterior surfaces 312, 316.

In one embodiment, device 300 also includes a transition zone between the nontransmissive portion 344 and the transmissive region 340. In one variation, the transition zone comprises an outer peripheral surface 352 of the transmissive region 340. The outer peripheral surface 352 can be configured to be nontransmissive, such as by disposing light absorbing particles or a coating on the surface 352. The nontransmissive portion 344 can thus provide sufficient space between the light that is transmitted through the transmissive portion 340 and the light that passes through the cornea around the ocular device to prevent the refractive difference between such light from producing noticeable glare. The transition zone can further depress or attenuate aberrant light effects, e.g., by providing an apodizing effect.

As discussed above in connection with the ocular device 100, the ocular device 300 can have recesses 360 that are configured to enable the ocular device 300 to maintain its position within the corneal, to transport nutrients, or to provide other advantages described herein.

### III. Ocular Devices for Compensating For Presbyopia

FIG. 8 illustrates an eye 10 that is presbyopic. Here, due to either an aberration in the cornea 14 or the intraocular lens 42, or loss of accommodation in the eye, for example due to age, light rays 32 entering the eye 10 and passing through the cornea 14 and the intraocular lens 42 converge a point behind the retina 22. The patient experiences this as blurred vision, particularly for up-close objects such as in reading. For such conditions, an ocular device 400 may be configured with a pin-hole aperture such that only a subset, e.g. a central portion, of light rays 32 is transmitted to the retina.

FIG. 9 shows the light transmission through an eye 10 that is presbyopic to which the ocular device 400 has been applied. Here, the light rays 32 that pass through the device 400, the cornea 14, and the lens 42 converge on the retina 22, e.g. at a single point. The light rays 32 that would not converge on retina 22, e.g. at the single point, are blocked by the device 400.

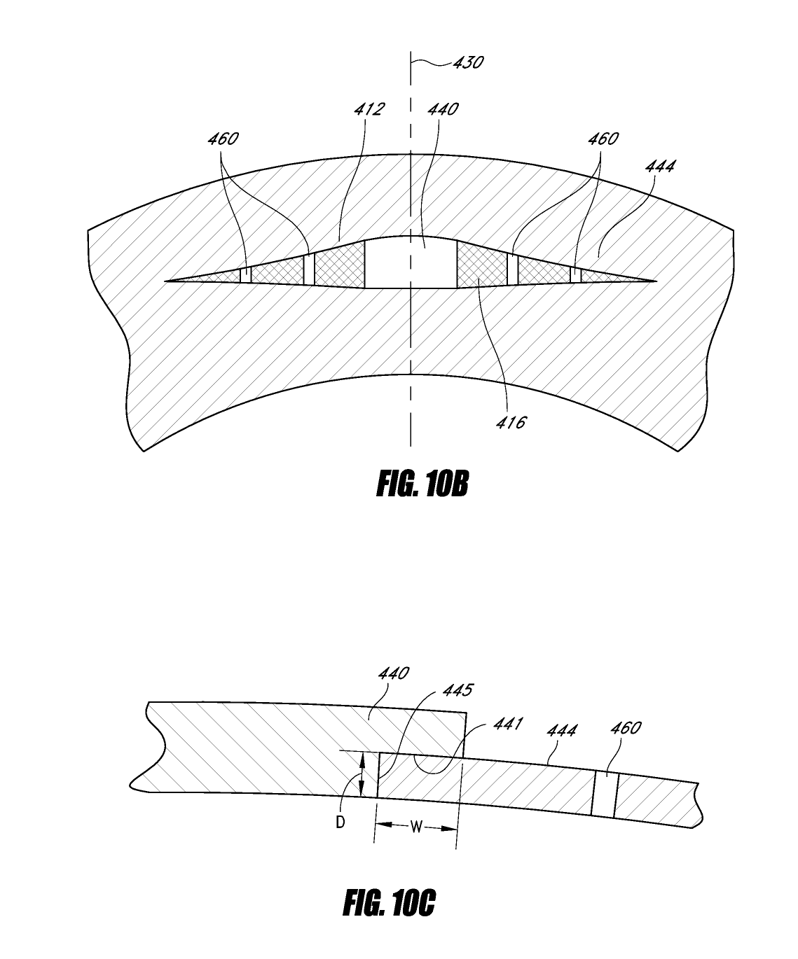

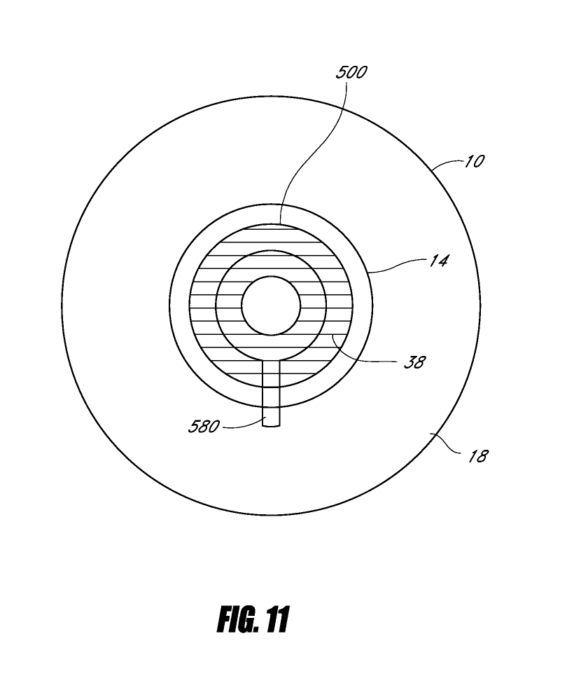

FIGS. 10A-B show further details of the ocular device 400, which can be used to improve the vision of patient with presbyopia. The ocular device 400 is similar to the ocular devices 100, 200 and 300, except as set forth below, and compatible structures of the ocular devices disclosed herein, e.g., the devices 100, 200, 300 and 400, can be interchanged. For example, the discussions above concerning materials and glucose transport properties and materials of the ocular device 100 also apply to the ocular device 400.

The ocular device 400 has a transmissive region 440 and an opaque region 444. The transmissive region 440 is smaller, e.g. having a smaller diameter, than the previously discussed ocular devices. In particular, the transmissive region 440 is small enough so that the device 400 operates as a stenopaeic aperture (e.g., creating a pinhole effect) in which only the central rays of light that would converge at or near the retina are transmitted. A substantial portion of the light rays that would not converge on or near the retina are not transmitted. The transmissive region 440 is preferably circular, e.g., surrounded by a circular boundary, and located about a central axis 430 of the device 400. In certain embodiments, the central axis 430 of the ocular device 400 coincides with the optical axis of the patient's eye. Techniques for aligning the central axis 430 of the device 400 with a patient's optical axis are discussed below.

As discussed above, the transmissive region 440 is configured to transmit substantially all visible light incident thereon. In one embodiment, a nontransmissive portion 444 surrounds at least a portion of the transmissive region 440 and substantially prevents transmission of incident light thereon. In one embodiment, the nontransmissive portion 444 comprises an annular mask extending peripherally from the transmissive region 440 toward an outer perimeter 408 of the device 400. The nontransmissive region 444 preferably completely surrounds the transmissive region 440. The nontransmissive region 444 is configured to block a substantial portion of visible light incident on an anterior surface thereof. In one embodiment, the nontransmissive region 440 blocks at least about eighty percent of the visible light incident on an anterior surface thereof. In another embodiment, the nontransmissive region 444 blocks ninety percent or more of the visible light incident on an anterior surface thereof In an alternative embodiment, the nontransmissive region transmits no more than twenty percent of the visible light incident thereon. As discussed above, the nontransmissive region 444 may be substantially opaque, or alternatively prevent transmission of the incident visible light by other optical phenomena such as one or more of reflection of light, diffraction of light, and scattering of light in the nontransmissive region 444, alone or in combination with light absorption. More details and variations on the nontransmissive region 444 and transmissive region 440 can be found in U.S. patent application Ser. Nos. 11/404,048, filed Apr. 13, 2006 and PCT Application No. PCT/US2010/045541, each of which is hereby incorporated by reference in their entirety.

As discussed above in connection with FIG. 8, preventing transmission of light through the nontransmissive portion 444 decreases the amount of light that reaches the retina that would not converge at the retina to form a sharp image. In the illustrated embodiment, the size of the transmissive region 440 is such that the light transmitted therethrough generally converges at the retina and a much sharper image is presented to the eye than would otherwise be the case without the device 400. Accordingly, the size of the transmissive region 440 may be any size that is effective to block the non-converging rays of light. By blocking the peripheral, non-converging rays, the pinhole increases the depth of focus of the patient's eye, thus increasing the depth of field (i.e. the range of distance along the optical axis in which an object can move without the image appearing to lose sharpness to the patient) of a patient suffering from presbyopia. In one embodiment, the transmissive region 440 can be circular, having a diameter of less than about 2.2 mm. In another embodiment, the diameter of the transmissive region 440 is between about 1.8 mm and about 2.2 mm. In another embodiment, the transmissive region 440 is circular and has a diameter of about 1.8 mm or less.

The transmissive region 440 may additionally have an optical power to compensate for a refractive error. The transmissive zone 440 can be arranged to provide a plus power of at least about 0.5 diopters in one embodiment. In another embodiment, the transmissive zone 440 can be arranged to provide a plus power of at least about 1.0 diopter. The optical power can be provided by modifying the curvature of the cornea or by providing a lens having a refractive power in the transmissive region. For example, as discussed above, the transmissive zone 440 can comprise a central lens portion made of a material having an index of refraction substantially different from the index of refraction of the corneal tissue. The refractive power of the lens may be selected to compensate for the mismatch between the refractive power of the eye and the length of the eye and thereby cause the transmitted light rays to properly converge on the retina. Such a lens could be configured with substantially the same curvature as that of a corresponding corneal layer, e.g., a layer that is adjacent to the transmissive zone 440 or to the anterior surface or posterior surface 412, 416. Alternatively, the lens portion may have a particular curvature, such as the biconvex lens, and positive and negative meniscus lens shown in FIGS. 7A-7C, that provides the necessary positive or negative power to correct for the refractive error of the patient's eye. In another embodiment, the skirt-like nontransmissive portion can have one or more ribs extending from the inner edge to the outer edge on the posterior side. The one or more ribs can be positioned to provide a change in the curvature of the cornea that provides the necessary positive or negative power to correct for the refractive error of the patient's eye. For example, in some embodiments, one or more ribs can extend radially from the transmissive region to create a steepening of the cornea when the implant is positioned therein and thus provide correction for hyperopia. Alternatively, one or more ribs can be placed annularly around the periphery of the nontransmissive portion to flatten the cornea when the implant is positioned therein and thus provide correction for myopia. In some embodiments, the one or more ribs can be used in conjunction with the shape and or thickness of the lens portion to produce the desired shape change in the cornea. In alternative embodiments, the one or more ribs may be positioned around the nontransmissive portion to provide a majority of the shape change to the cornea. Here, the lens portion can be optically clear or alternatively, the lens portion can be removed altogether.

In one embodiment, the transmissive zone 440 has a lens structure in which at least one of the anterior and posterior surfaces is spherical. In one arrangement, the transmissive zone 440 has a posterior surface that has a first radius of curvature and the anterior surface that has a second radius of curvature. The first and second radiuses can be substantially equal or can be different. In one embodiment where it is desired to substantially maintain the curvature of the anterior surface of the cornea, the anterior surface of the transmissive zone 440 is configured to correspond to, e.g., is matched with or substantially identical to, the curvature of the anterior surface of the cornea. In one embodiment where it is desired to substantially maintain the curvature of the posterior surface of the cornea, the posterior surface of the transmissive zone 440 can be configured to correspond to, e.g., is matched with or substantially identical to, the posterior surface curvature of the cornea. In another embodiment, both of the anterior and posterior surfaces of the transmissive zone 440 are configured to correspond to the anterior and posterior surfaces of the cornea such that the curvature of the cornea is substantially the same after the device is implanted as before implantation thereof. In this context “substantially the same” include conditions where curvature of the cornea is modified to some extent, but changes in power of the eye due to such curvature changes do not noticeably contribute to power change of the eye (though other factor such as index of refraction might change the power).

In some embodiments, the curvature of at least one surface of the transmissive zone 440 is specifically mismatched from a corresponding surface of the cornea. For example, the anterior surface curvature of the transmissive zone 440 can be selected to be sufficiently different from the anterior surface curvature of the cornea to induce a power changing curvature change of the anterior surface. In another embodiment, the posterior surface curvature of the transmissive zone 440 can be selected to be sufficiently different from the posterior surface curvature of the cornea to induce a power changing curvature change of the posterior surface. In some embodiments, both posterior and anterior curvatures of the transmissive zone 440 are selected to be sufficiently different from the corresponding posterior and anterior corneal surface curvature of the patient's eye to produce a desired ocular power or power change. In some embodiments, the curvature of at least one surface is specifically mismatched in one direction to induce a cylinder power for correction of astigmatism.

In one embodiment, the transmissive zone 440 has a spherical anterior surface and a spherical posterior surface. The anterior surface of the transmissive zone 440 can have a radius of curvature of about 7.5 mm in one embodiment. In one variation, the anterior surface curvature of the transmissive zone 440 is about 7.0 mm. In another variation, the anterior surface curvature of the transmissive zone 440 is about 6.5 mm. The posterior surface of the transmissive zone 440 can have a curvature of about 7.35 mm in one embodiment. The transmissive zone 440 can have any suitable thickness. For example, in one embodiment, the transmissive zone 440 is about 50 microns thick. In another embodiment, the transmissive zone 440 is less than about 50 microns thick at its thickest point. In another embodiment, the transmissive zone 440 between about 50 microns thick and about 100 microns thick at its thickest point. In some applications, the transportability of a nutrient across the transmissive zone 440 is an important parameter. For example, it is desirable to configure the transmissive zone 440 to not deprive tissue adjacent thereto of glucose. Accordingly, it is desirable to increase the nutrient transporting capabilities of the transmissive zone 440 as the transmissive zone is made thicker.

FIG. 10C illustrates that in one embodiment, the ocular device 400 can be configured to provide a mechanical coupling of the transmissive zone 440 with the nontransmissive zone 444. In particular, in one embodiment, a recess 441 is formed in the outer periphery of the transmissive zone 440. The recess 441 can be in form of a peripheral shelf that can extend a portion of or all the way around the outer periphery of the transmissive zone 440. As discussed herein, in some embodiments, the transmissive zone 440 can be configured to increase in volume or in at least one dimension such as transverse size when in an aqueous environment. In one embodiment, the recess 441 can be positioned within an inner periphery 445 of the nontransmissive zone 444 in a partially or un-hydrated condition. Thereafter, the transmissive zone 440 can be exposed to a liquid, such as water or saline, to become more fully hydrated. As the transmissive zone 440 absorbs the liquid, it swells in some embodiments, such that the recess 441 is brought to bear upon the inner periphery 445 of the nontransmissive zone 444. This sort of assembly can be performed during the manufacturing process, in pre-operative preparation, or during the procedure, such as on the cornea or an exposed internal layer thereof.

In one embodiment, the transmissive zone 440 has a transverse dimension, e.g., a diameter if the transmissive zone 440 is circular, of between about 1.1 and about 1.2 mm. The transmissive zone 440 can have a diameter of about 1.18 mm. In one embodiment, the transmissive zone 440 has a transverse dimension, e.g., a diameter if the transmissive zone 440 is circular, of between about 1.2 and about 1.8 mm. In one embodiment, the transmissive zone 440 has a diameter of about 1.6 mm. The transmissive zone 440 can have a diameter of about 1.35 mm. In one embodiment a peripheral shelf is provided between the outer periphery of a first surface of the transmissive zone 440 and the outer periphery of a second surface of the transmissive zone 440. For example, the posterior surface can have a diameter of about 1.2 mm, the anterior surface can have a diameter of about 1.35 mm providing a peripheral shelf therebetween having a width, W, of about 0.075 mm. The peripheral shelf can be annular, including extending all the way around the transmissive zone. The shelf also can have a suitable depth, D, relative to the posterior surface. In one embodiment the depth of the shelf is about one-half of the thickness of the transmissive zone 440. In one embodiment the depth of the shelf is about 0.02 mm. However, any suitable shelf depth can be provided that enables the nontransmissive portion 444 to adequately couple with the transmissive portion 440 where a mechanical coupling of these components is desired. In some embodiments, the shelf is located on the anterior side of the device such that the anterior surface of the transmissive portion 440 has a smaller diameter than the posterior surface.

As shown in FIG. 10B, the ocular device 400 can be configured in any manner described above in connection with the ocular device 100 to be positioned between layers of the cornea 14 of an eye 12. At least one of the anterior and posterior surfaces 412, 416 of the ocular device 400 can be configured to reside adjacent to or to abut corneal tissue. In one variation, the anterior surface 412 may have a curvature that is similar to the curvature of a first corneal layer. The posterior surface 416 may also have a curvature similar to the curvature of the second corneal layer, or alternatively, may be configured to provide a positive or negative power, for compensating for a refractive error of the eye. In certain embodiments, the curvature of the posterior surface 416 may be configured to alter the curvature of the posterior corneal layer, thereby providing additional refractive correction.

As discussed above in connection with the ocular device 100, the ocular device 400 may have a plurality of recesses 460 extending from the anterior surface 412 toward the posterior surface 416. The recesses 460 can be configured to enable the ocular device 400 to sufficiently grip adjacent corneal tissue such that the ocular device 400 will not migrate or rotate in the eye after implantation. In some embodiments, the recesses 460 provide nutrient transfer between the adjacent layers of the corneal tissue. In some embodiments, the recesses are configured to sufficiently grip adjacent corneal tissue such that the ocular device will not migrate or rotate in the eye after implantation and to provide nutrient transfer between the adjacent layers of the corneal tissue. The recesses 460 can be similar to the recesses 160.

### IV. Ocular Devices Comprising a Locator Structure

Certain embodiments may further include a locator structure that indicates the location of (e.g., the depth of) the implant within the eye when implanted. Examples of such locator structures are disclosed in co-pending application U.S. application Ser. No. 11/106,040, entitled “Ocular Inlay with locator,” filed on Apr. 15, 2005, the entirety of which is hereby incorporated by reference. Normal healing processes result in the incisions being sealed, making the location of the implant difficult to find. Thus, a locator structure may be used to facilitate locating the implant once it has been implanted. The locator structure can extend radially from the implant, as discussed further below. The locator structure may also or alternatively be utilized to facilitate removal of the ocular device from the eye. The various forms of locator structures discussed below can be used in connection with methods, techniques and procedures for removing an inlay or mask that has been applied in any manner discussed below or in any other suitable manner.

The locator structure may comprise any of a wide variety of configurations, such as radially outwardly extending flanges, tabs, loops or tethers, depending upon the desired clinical performance. In general, the locator structure will extend radially outwardly from the periphery of the implant for a distance sufficient to extend outside of the patient's line of sight. In certain embodiments, the length of the locator structure from a periphery of the implant will be at least about 25%, in some embodiments at least about 50%, and in other embodiments at least about 75% or 100% or more of the diameter of the implant. In some embodiments, the locator structure is an unobtrusive structure that is visible or is made visible only to clinical personal during an ocular procedure.

FIG. 11 shows an implant 500 implanted generally centrally in the eye 10 and at a selected layer of the cornea 14. It should be understood that FIG. 11 is schematic in nature and should not be interpreted as being strictly to scale, however showing generally the eye 10 including the cornea 14 and the pupil 38. The implant may include at least some of the features of or may be similar to any of the ocular devices disclosed herein. In the illustrated embodiment, the implant 500 includes a stenopaeic aperture and a lens, similar to the ocular device 400. However, the locator structure discussed below in connection with the ocular device 500 is also applicable to the ocular structure 100, which does not have a stenopaeic opening in the illustrated embodiment. The implant 500 preferably includes a locator structure 580 that is configured to facilitate locating the inlay assembly 500 after implantation. Normal healing processes result in the incisions being sealed, making the location of the inlay assembly 500 difficult to find. As discussed further below, the locator structures 580 make the inlay assembly 500 easier to find and may be used to facilitate removal of the implant

In the illustrated embodiment, the locator structure 580 comprises an elongate tail-like member that extends from a periphery of the implant 500. The tail-like member is long enough to extend at least partly beyond the pupil region. Although the illustrated locator structure 580 is configured as a radially outwardly extending tab, having a substantially uniform cross section along its length, and a width of less than about 25% of the diameter of the inlay assembly 500, any of a variety of alternative structures may be utilized. For example, locator structure 580 may comprise a tether, such as a single strand or multi-strand filament, extending from the inlay assembly 500 and provided with a free end, which may be formed into a loop or eye to facilitate grasping by a removal tool. Alternatively, the locator structure 580 may comprise a strip or band or filament that extends in a closed loop, being attached to the inlay assembly 500 at two points. This provides a loop or handle which may facilitate grasping by a removal tool. In certain embodiments two, three or more locator structures may be attached to the implant, depending upon the desired clinical performance.

The locator structure 580 may either be formed integrally with the inlay assembly 500, or may be formed separately and secured to the inlay assembly 500 as a separate step. Any of a variety of attachment techniques may be utilized, depending upon the construction materials for the inlay assembly 500 and the locator structure 580, such as thermal bonding, adhesive bonding, chemical bonding, interference fit, or other techniques known in the art. Any of a variety of techniques which are known presently in the art for attaching haptics to an intraocular lens may also be used.

The locator structure 580 may comprise the same material as the implant 500, or any of a variety of implantable materials known in the art, such as polypropylene, polyethylene, polyimide, PEEK, Nylon, and a variety of biocompatible metals such as stainless steel, Nitinol or others depending upon the desired performance of the implant.

In certain embodiments, the locator structures may be configured to be visible under normal direct visualization. For example, opaque or partially opaque locator structures may accomplish this objective, such as through the use of metals or polymers having a dye or other constituent which absorbs light in the visible range. However, the cosmetic result may be undesirable, and other location techniques may be preferred. An optically transparent locator structure may be located by tactile feedback, such as through the use of a small probe.

Alternatively, the locator structure may comprise a tail having a marker region positioned on the distal end of the tail and a transparent region located at an intermediate or proximal end of the tail. In certain embodiments, the marker region may be provided with a tinting or coating such that the marker region exhibits increased contrast with background/underlying eye tissue to facilitate identification and location of the ocular device. In alternative embodiments, the marker region may be formed with selected dye materials such that illumination with electromagnetic radiation of selected wavelengths induces the marker region to disproportionately luminance or fluoresce in the visible light range such that under selected observation conditions the marker region exhibits enhanced contrast against adjacent tissue. Thus, the locator structure may be unobtrusive and substantially invisible under normal casual observation conditions, but, is readily visible under selected artificial viewing conditions to facilitate location of a selected level or depth of the eye in which the inlay assembly is implanted.

In use, the locator structure may be implanted in a therapeutic location at least partially overlapping the pupil region 34 at a selected level of the patient's eye 10. Thus, following implantation and healing processes, a physician could identify and locate the selected level at which the locator structure, and thus the ocular device, is positioned and after identifying the selected level, proceed at that level to access the ocular device, for example for removal and replacement.

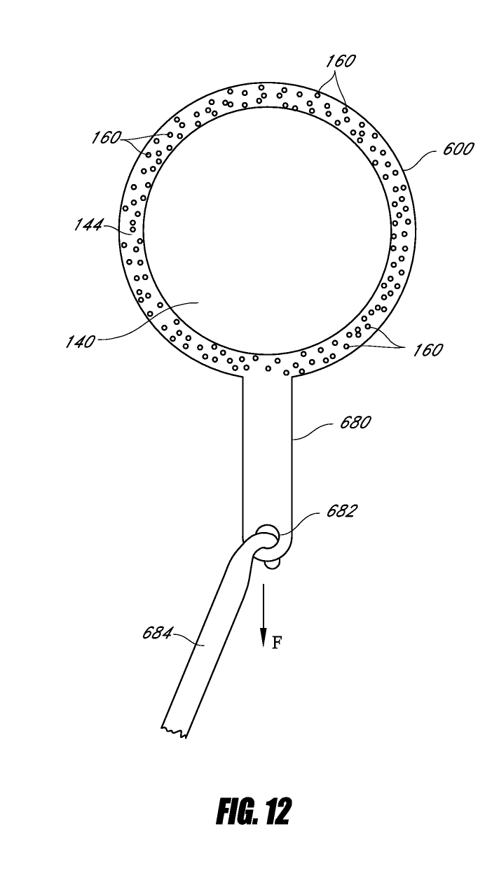

FIG. 12 illustrates another embodiment of an implant 600 comprising a locator structure 680 and a retrieval structure 682. In general, the retrieval structure 682 comprises at least one transverse engagement surface to facilitate engagement by a retrieval instrument 684. The engagement surface can be provided in any of a variety of ways. For example, in the illustrated embodiment, the retrieval structure 682 comprises a single aperture formed in a distal portion of the locator structure 680. Alternatively, two or three or four or more apertures may be provided in the locator structure 680. The retrieval structure 682 may alternatively be formed by attaching the locator structure 680 at 2 points to the inlay assembly 600, to produce a loop or handle configuration. In this configuration, the transverse retrieval surface is formed on the surface of the locator structure facing the inlay assembly 600. Any of a variety of alternative retrieval structures 682 may be provided, depending upon the desired clinical performance, such as providing the locator structure 680 with texturing, one or more ridges or corrugations, friction enhancing surfaces, or other structure, depending upon the desired cooperation with the complementary surface structures on the desired retrieval tool.

Additional details of particular embodiments of locator structures which may be advantageously utilized with the ocular devices described herein are described in greater detail in U.S. patent application Ser. No. 11/106,040, filed Apr. 14, 2005 and entitled “OCULAR INLAY WITH LOCATOR” and in U.S. patent application Ser. No. 11/106,043, filed Apr. 14, 2005 and entitled “CORNEAL OPTIC FORMED OF DEGRADATION RESISTANT POLYMER” and in U.S. application Ser. No. 11/107,359 entitled “METHOD OF MAKING AN OCULAR IMPLANT” filed Apr. 14, 2005, all of which are incorporated herein in their entirety by reference.

### V. Methods of Implanting Ocular Devices

As discussed above, any of the ocular devices disclosed herein can be coupled with a cornea using a variety of suitable techniques. Such techniques can include forming a flap of corneal tissue to expose first and second corneal layers, forming a pocket within the cornea, and creating a cavity within the cornea. These techniques are discussed below in connection with the ocular device 100, but are also applicable to the other ocular devices disclosed herein.

### A. Techniques for Implanting an Ocular Device Under a Flap

Adjacent layers of the stroma may be accessed by creating a flap in a variety of ways in connection with implanting the ocular device 100. A suitable technique of creating a flap to expose a layer of the cornea between the epithelium and the endothelium is shown with reference to FIGS. 13A-13B. Preferably, in creating the flap 716, first and second corneal layers can be exposed. The location of the first and second layers can be any desirable depth within the cornea. For example, in one technique, the first and second layers are located at between 100 and 300 microns depth as measured from the anterior surface of the cornea. In one technique, the first and second layers are between about 150 and about 250 microns in depth. In another technique, the first and second layers are at about 200 microns depth within the cornea. Similar depths can be accessed through pocketing or laser-cavity forming techniques discussed below. The first and second corneal layers can be layers that are normally adjacent to each other with the first corneal layer 715 being on the flap 716 and the second corneal layer 717 being the exposed, anterior-most layer of the stroma or of the cornea 714 when the flap 716 is peeled back. In some techniques, as discussed further below, it is desirable to additionally remove some tissue to form a recess so that the ocular device 100 can be accommodated without substantial change in the shape of the cornea 14.

To form a flap 716, a cutting implement can be used to create an incision. The cutting implement can take any suitable form. In one technique, a microkeratome or a laser is used to form an incision. The laser can be a femtosecond laser in some embodiments. The incision can be arcuate in shape, e.g., circular. In a flap technique, a layer of tissue can be fully removed from the eye or the tissue layer can be attached along a small arc of the circular. Thereafter the tissue forming the flap 716 can be removed or peeled back from the eye to expose at least one of the first layer 715 and the second layer 717. Thereafter, corneal tissue can be removed if desired to expose another layer and to create a volume within the cornea within which the ocular device 100 can be placed.

In connection with the flap technique, the medical professional performing the procedure can employ a technique for centering the ocular device relative to an ocular feature. The feature can be a visible ocular feature such as a pupil, sclera, or a portion of an iris, a mark on the cornea, or an ocular feature that is not visible, such as the patient's line of sight. The ocular device can be placed on either the second (exposed) layer 717 on the cornea 14 or the first layer 715 on the flap during or after the centration process. Thereafter, as shown in FIG. 13B, the flap 16 can be placed back on the remaining portion of the cornea 14 with the ocular device 100 sandwiched between the tissue forming the flap and the remaining portion of the cornea.

As discussed above, after the cornea has been replaced over the top of the ocular device 100 the curvature of the anterior surface of the cornea can be altered. In some cases, the change in curvature is minor and the changed curvature does not impart a significant change in the optical performance of the eye. In other cases, the ocular device 100 is configured to produce a noticeable change in the curvature of the eye, e.g., to produce enough of a change in the optical performance of the eye to impart a corrective effect. In some examples, the curvature change imparted by the ocular device 100 (or by any of the other ocular devices described herein) can cause a steepening or a flattening of the curvature of the cornea which can provide a refractive correction in vision.

### B. Techniques for Implanting an Ocular Device in a Pocket

Although flap techniques are a convenient manner for implanting the ocular devices disclosed herein, such devices can also be deployed in an eye by making a smaller incision in the anterior surface of the cornea and creating a corneal pocket through the small incision, for example by delaminating adjacent layers of corneal tissue.

With reference to FIGS. 14A-D, a pocket can be made through a small incision using a hand tool whereby enough space to receive the ocular device 100 can be created in a pocket. In particular, an incision 800 can be made in the anterior surface of the cornea 14. The incision 800 can be made in any suitable manner, such as with a microkeratome or a laser (e.g., a femtosecond laser). Thereafter, a space, or pocket 802 can be created between adjacent layers of the cornea. As shown in FIG. 14B and FIG. 14C, two adjacent corneal layers can be separated using a thin implement 804 adapted to delaminate corneal tissue. The pocket 802 formed within the cornea can have a transverse dimension that, at its maximum, is larger than the width of the incision 800. More details of one form of this technique are set forth in U.S. Pat. No. 4,607,617, issued Aug. 26, 1986 and in U.S. Pat. No. 4,655,774, issued Apr. 7, 1987 which are hereby incorporated by reference herein.

FIGS. 14A-E illustrate various techniques for forming a corneal pocket using manual manipulation of surgical instruments, other techniques for forming a corneal pocket can incorporate the use of automated pocket making tools. These automated or automatic tools for making pockets can include a structure that immobilizes the cornea relative to the tool and a blade that travels in a predetermined profile to create a corneal pocket of desired size and shape. For example, the blade can follow a profile to form a corneal pocket that dimensionally closely matches the ocular device 100. With this close dimensional matching, the corneal pocketing procedure can minimize the trauma and/or impact on the corneal tissue. Also, close dimensional matching of the pocket and implant sizes, alone or in combination with small gripping holes, can help retain the ocular device 100 once implanted. Examples of manual and automated pocket making tools are set forth in U.S. Pat. No. 5,964,776, issued Oct. 12, 1999, and U.S. Patent Application Publication No. 2005/0049621, published Mar. 3, 2005, both of which are hereby incorporated by reference herein.

The size of the incision 800 can be about equal to a transverse dimension of the ocular device 100 or somewhat larger in one technique so that the ocular device can be inserted in a flat configuration. In one technique, where the ocular device 100 is formed of a material that can be rolled or folded, the incision 800 providing access to the pocket 802 can be smaller than a transverse dimension of the ocular device 100. This can be accomplished by swinging a distal portion of a pocket creating implement (e.g. a pocket creating tool) through an arc centered near the incision 800. A pocket in which the incision width and pocket width are closely matched can be accomplished by a transverse movement of an implement as illustrated in FIG. 14C. In one technique, the transverse size of the incision 800 is a fraction of the transverse size of the pocket 802. In particular, the transverse size of the incision 800 can be about one-half the transverse size of the pocket 802 or less in one technique. In another technique, the transverse size of the incision 800 can be about one-third the transverse size of the pocket 802 or less. In another technique, the transverse size of the incision 800 can be less than about one-quarter the transverse size of the pocket 802. Advantageously, where the incision 800 is smaller than the transverse dimension of the ocular device 100, interference between the incision 800 and the ocular device 100 tends to restrain the ocular device 100 within the cornea once implanted.

After the pocket 802 has been formed, the ocular device 100 can be implanted moved into and positioned within the pocket 802 as shown in FIG. 14D-E. If the ocular device is implanted in a flat configuration, the implant is advanced distally into the pocket in the manner illustrated in FIG. 14D-E. This can be accomplished by using an insertion tool 810, which can be configured with anterior and posterior fork elements 812 or can be configured with anterior and posterior loop elements 814. The anterior and posterior fork element 812 can be configured to grasp or support at least one of an anterior surface and a posterior surface of the ocular device 100. Once in the pocket, the ocular device 100 can be positioned to a selected position, e.g., to a position corresponding to a visible ocular feature. In may be desired, for example, to align or closely position an optical axis of the ocular device 100 and the line of sight of the patient, in any suitable manner, as discussed above. Thereafter, the incision 800 can be closed in a suitable manner.

More details relating to techniques for implanting any of the ocular devices 100, 200, 300, 400, 500, and 600 are discussed in U.S. patent application Ser. No. 10/854,033, filed on May 26, 2004 and entitled “MASK CONFIGURED TO MAINTAIN NUTRIENT TRANSPORT WITHOUT PRODUCING VISIBLE DIFFRACTION PATTERNS”, which is incorporated by reference in entirety.

### C. Techniques for Implanting an Ocular Device in a Cavity

In addition to the techniques discussed above, a further step can be performed in which a cavity is formed or enlarged within the cornea. Such a cavity can be conveniently formed or enlarged using a laser, such as a femtosecond laser. Although a microkeratome could be used to create or enlarge a cavity, a laser is particularly convenient in that the step can be performed prior to an incision being made in the anterior surface of the cornea. That is, such a laser technique could form the cavity by being focused at a discrete location, e.g., at a selected depth in the cornea, through one or more layers of the cornea. Preferably the dimensions of the cavity formed or enlarged can be selected in accordance with the configuration of the implant.

After the laser has been used to form the cavity, an access path can be provided from the anterior surface of the cornea to the cavity. For example, an incision can be made in the cornea and an access path can be formed from the incision to the cavity. The incision preferably is formed at a peripheral location of the anterior surface of the cornea and the access path extends from the incision to a selected location on the cavity, e.g., to a peripheral portion of the cavity. The incision or the access path can be any suitable size, e.g., can be sized to permit the corneal implant to be delivered to the cavity in the same configuration in which it is applied. Alternatively, the incision and/or the access path can be formed to minimize tissue disruption. Where tissue disruption is desirable, the ocular device can be delivered in a low profile configuration, e.g., compacted or rolled or in another suitable low profile configuration.

One advantageous way to access the cavity is by making a self-sealing incision. A self-healing incision can be formed in the eye at a location outside of the optical zone. The limbus is one location where this type of incision can be made. After the limbal incision is made, an angled pathway can be formed between the limbal incision and a location in the stroma of the cornea corresponding to the depth at which the ocular device is to be placed. In one technique, a pocket will have been created prior to forming the limbal incision. The pocket can then be accessed through this layer of the stroma. Preferably the angled pathway is extended from the limbal incision to the stromal layer where the pocket had been created. In another technique, a pocket can be formed after the limbal incision is made an the corneal layer is accessed through the angled pathway. The self-sealing incision is one that will heal without significant postoperative intervention by the surgeon. Preferably stitches or other closure devices are not needed, but the normal intraocular pressure causes the opposing sides of the incision to be urged into sealing engagement. Eventually, the incision becomes covered by epithelium.

More details relating to techniques for implanting any of the ocular devices described herein are discussed in U.S. patent application Ser. No. 10/854,033, incorporated herein by reference above.

D. Techniques for Aligning an Implant with the Optical Axis of a Patient's Eye

Alignment of the central transmissive region of the ocular devices disclosed herein with the visual axis of the patient is believed to provide greater clinical benefit to the patient. The eye orients itself so that an object being viewed is centered on the visual axis, which causes light rays from the object to be focused on the fovea, as discussed above. Although the ocular devices disclosed herein can work in a variety of positions, it is preferred to that devices be aligned with the visual axis of the eye such that the visual axis extends through the central transmissive region of the ocular device. Thus, the refractive power of the ocular device can act upon the light rays from the object being viewed in the proper manner. The visual axis of the eye is not necessarily located at the center of the pupil. Accordingly, any of a variety of techniques to locate the visual axis can be used to aid in implanting the ocular devices disclosed herein.

The patient's visual axis may be located in a variety of ways such as using a pharmacological agent. The pharmacological agent can be applied to the patient. In one technique, a pupil constricting drug, such as pilocarpine or any other suitable drug, is applied to the patient's eye to cause the pupil to restrict. The diameter and location of the pupil may first be measured in its unrestricted state and then again after application of the drug in its restricted state. Comparison of the pupil in its constricted and unconstricted state will show the general location of the patient's visual axis. Once the patient's visual axis has been determined, the optical axis of the ocular device may be aligned with or positioned near the patient's visual axis. Such methods are further explained in co-pending U.S. patent application Ser. No. 11/257,505, filed on Oct. 24, 2005 and entitled “SYSTEM AND METHOD FOR ALIGNING AN OPTIC WITH AN AXIS OF AN EYE,” hereby incorporated by reference in its entirety. Alternately, masks may be applied to the surface of the cornea and later used to align the device.

### VI. Methods of Making Ocular Devices

FIGS. 15-17 illustrate various methods for making ocular devices with nontransmissive (e.g., opaque) portions and transmissive portions. In some techniques, an opaque portion is formed first and a transmissive portion is formed thereafter. In some techniques, a transmissive portion is formed first and an opaque portion is formed thereafter.

FIG. 15 shows an embodiment of an ocular device 900 that is suitable for implantation between layers of a cornea of an eye. The ocular device 900 can be made by forming a transmissive portion 940 within an opaque portion 944. The opaque portion 944 of the ocular device 900 can be pre-formed using any suitable process. Alternately, the opaque portion and the transmissive portion 940 can be formed together as discussed further below. The opaque portion 944 can include a plurality of recesses similar to those hereinbefore described. The opaque portion 944 can have any of the properties of corresponding portions of any of the ocular devices disclosed herein.

In a first technique, a material forming the transmissive portion 940 of the ocular device 900 is disposed within the opaque portion 944. In one variation of this process, the material forming the transmissive portion 940 is a material that has a contracted or a low-volume configuration prior to be disposed within the opaque portion 944, e.g., within an inner periphery 946, and has an expanded or high volume configuration thereafter. A material that absorbs a liquid to expand in this manner would be suitable. For example, in certain embodiments, a hydrogel could be used to form the transmissive portion 940. Once the transmissive portion has been placed within the inner periphery 946 of the opaque portion 944, the transmissive portion 940 preferably expands into secure engagement with the inner periphery 946.

In combination with the foregoing technique, a further technique can be used to further configure the transmissive portion 940 to refract light to compensate for refractive error. For example, the transmissive portion 940 can be shaped in a suitable manner, e.g., having a selected convexity or concavity on at least one of the anterior and posterior surfaces thereof to provide positive or negative optical power to the transmissive portion for correcting a refractive error of the eye.

A variation on the foregoing technique is to form the transmissive portion 940 to be capable of transporting nutrients from a posterior side to an anterior side. One approach to making the transmissive portion 940 capable of such transmission is to provide pores or internal channels through which the nutrients can pass. Such pores or internal channels can be formed in a process that involves forming a material or substance that includes a network of absorbent polymer chains and a diluent. The network of polymer chains can be of a biocompatible hydrogel material. The diluent in the material or substance can be absorbed by or disposed within the network of polymer chains. The diluent can be any suitable material, such as one that is water soluble. It is anticipated that a biocompatible diluent would be particularly advantageous. For example, in some embodiments, the diluent could be poly ethylene glycol (PEG) or heparin. However, a less biocompatible material could be used in certain embodiments, as discussed further below. In alternative embodiments, the diluent can advantageously be a wound healing modulator compound such as hyaluronic acid or any other suitable wound healing agent. A variety of other wound healing agents are set forth in U.S. patent application Ser. No. 11/404,048, filed on Apr. 13, 2006, which is hereby incorporated by reference herein. The diluent can have any of a variety of molecular weights, e.g., of a few thousand up to about two-hundred to three hundred thousand Daltons. In one technique, the diluent is a material that will migrate out of the pores or channels in the polymer network and be replaced by a liquid such as water or saline when exposed to such liquid. One mechanism for transfer of the diluent out of the pores or channels is concentration gradient. For example, upon exposure to an exchange liquid such as water or saline, the diluent will flow from the polymer chains to minimize the concentration gradient between the polymer chain and the surrounding exchange liquid. In some embodiments, a sufficient volume of exchange fluid may be flushed over the transmission portion for a sufficient length of time to exchange substantially all of the diluent for the exchange fluid. This technique of forming the transmissive portion 940 with a diluent has the advantage of permitting the transmissive portion 940 to have a manufactured volume that is similar to the deployed volume in the cornea.

In another method, a lens formed by diluent exchange can be coupled with an annular mask portion comprised of a second material. The second material is different from the first material.

FIG. 16 illustrates schematically a second technique that could be used to form the transmissive portion 940 in a preformed opaque portion 444. In the second technique, a mold 950 is provided that has a first portion 952 into which the opaque portion 944 can be disposed. In one technique, a second portion 954 of the mold 950 is configured to couple with the first portion 952. The first and second portions 952, 954 can be shaped to form the transmissive portion 940 in a manner that enables the ocular device 900 to refract light to compensate for refractive error of the eye. Preferably at least one of the first and second portions 952, 954 is transmissive to electromagnetic radiation, e.g., ultraviolet light, which can be used to solidify the transmissive portion 940 in one technique.

A material that will form the transmissive portion 940, e.g., one that provides suitable refractive and transmissive properties and that can be made solid by exposure to the electromagnetic radiation, is thereafter disposed in the mold 950. In one technique, the opaque portion 944 is disposed in the mold after the material that will form the transmissive portion 940. The mold 950 can thereafter be exposed to electromagnetic radiation to form the transmissive portion 940. In some cases, the ocular device 900 is further processed thereafter, e.g., refining the shape of at least one of a posterior and an anterior surface thereof. The mold 950 advantageously can be used to form the transmissive portion 940 in any suitable shape, e.g., with at least one convex or concave surface, bi-convex, bi-concave, or any other combination of surfaces.

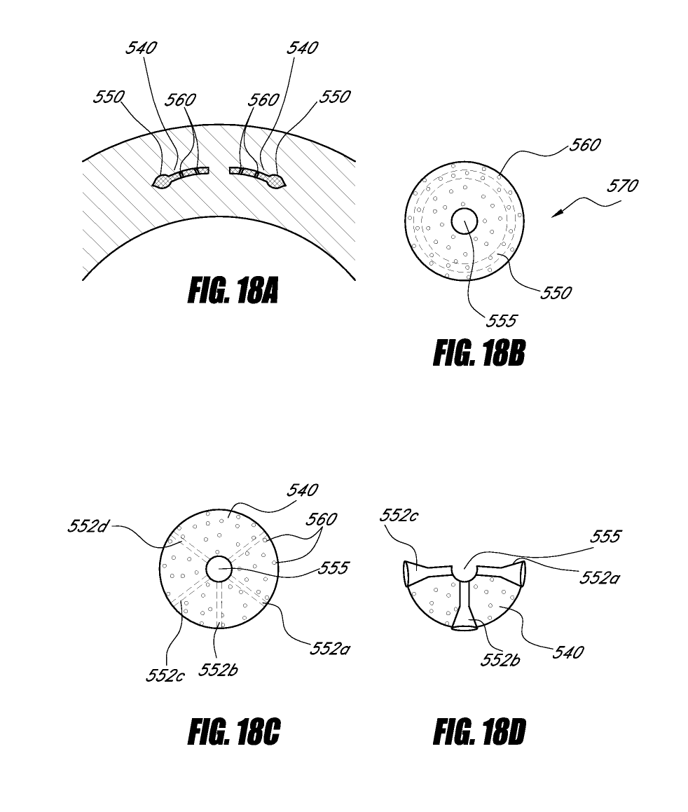

FIGS. 17A-17C illustrate another method of forming an ocular device 1000. In this method a mold 1050 is formed. The mold has a central portion 1052 and an annular portion 1054. A preformed transmissive portion 1040 is positioned in the central portion 1052. Thereafter, an opaque portion 1044 can be formed by flowing a liquid into the annular region 1054 of the mold 1050. The liquid can be a combination of suitable materials, such as PVDF, or another material resistant to UV degradation, or another suitable base material and carbon particles, or another suitable opacification agent. The opaque portion 1044 can then be caused to solidify. In certain embodiments, the mold 1050 can be configured to produces the ocular device 1000 in an implantable form. Alternatively, as illustrated, the mold 1050 may produce a structure 1002 including an unshaped transmissive portion 1040 and an unshaped opaque portion 1044 that is a unitary, solid construction but which is not fully shaped. Thereafter, any suitable technique can be used to shape the structure 1002 into the ocular device 1000, e.g., having surfaces that conform to natural corneal curvature, or that produce a refractive effect that compensates for refractive error of the eye. FIG. 17C illustrates one embodiment of the fully formed ocular device 1000, having concave anterior and posterior surfaces. FIG. 17C also illustrates that a plurality of recesses 1060 can be formed in the ocular device 1000 after being molded as shown in FIG. 17A. In some embodiments, the structure 1002 can be implantable without further shaping, e.g., where it is intended to confirm to the natural curvature of the patient's eye, e.g., providing refractive compensation by the refractive index of the material used. Alternatively, the implant 1000 may be further shaped on the anterior or posterior surfaces to provide for a positive or negative lens, or alternatively to provide refractive correction by modifying the curvature of the cornea once implanted. Additional methods and techniques to form an ocular device are further explained in U.S. patent application Ser. No. 12/856,492, filed on Aug. 13, 2010, hereby incorporated by reference in its entirety.

### VII. Occular Devices Comprising a Rib Structure

FIGS. 18A-D illustrate additional embodiments of an ocular device 570 which can be used to improve the vision of a patient with presbyopia. The ocular device 570 is similar to ocular devices 100, 200, 300 and 400 except as set forth below and compatible structures of the ocular devices disclosed herein, e.g. 100, 200, 300 and 400, can be interchanged.

The ocular device 570 has an annular non-transmissive region 540 surrounding a stenopaeic opening or aperture 555 which creates a pin-hole effect. In certain embodiments, the aperture 555 is located about a central axis of the ocular device 55 and may coincide with the optical axis of the patient's eye. The skirt-like, nontransmissive region 540 can be substantially opaque and can be configured to block a substantial portion of light incident on the anterior surface thereof. In certain embodiments, the non-transmissive region can be color matched to the patient's pupil, or alternatively can be made black using the techniques described above. In certain embodiments, the non-transmissive region can include a plurality of recesses 560.

As discussed above, preventing transmission of light through the nontransmissive portion 540 decreases the amount of light that reaches the retina that would not converge at the retina to form a sharp image. In certain embodiments, the size of the aperture 555 is such that the light transmitted therethrough generally converges at the retina and a much sharper image is presented to the eye than would otherwise be the case without the device 570. Accordingly, the size of the aperture 555 may be any size that is effective to block the non-converging rays of light. By blocking the peripheral, non-converging rays, the aperture 555 increases the depth of field (e.g. the range of distance along the optical axis in which an object can be moved without the image appearing to lose sharpness). For example, the aperture 555 can increase the depth of field of a patient suffering from presbyopia. In one embodiment, the aperture 555 can be circular, having a diameter of less than about 2.2 mm. In another embodiment, the diameter of the aperture 555 is between about 1.8 mm and about 2.2 mm. In another embodiment, the aperture 555 is circular and has a diameter of about 1.8 mm or less.

In certain embodiments, the nontransmissive region 540 includes one or more ribs to provide a change in the curvature of the cornea to correct for the refractive error of the patient's eye. In certain embodiments, the ribs are positioned on the posterior side and/or the anterior side of the nontransmissive region 540. The number of ribs, position of the ribs on the non-transmissive region and thickness of the ribs can be varied to provide adjustments to the curvature of the cornea to provide refractive correction.

In certain embodiments, one or more ribs 550 can be placed annularly around the periphery of the nontransmissive portion 540 to flatten the cornea and thereby provide refractive correction for myopia or hyperopia, as illustrated in FIGS. 18A-B. The location of the annular rib 550 may can be within the outer periphery of the nontransmissive portion 540, as illustrated in FIGS. 18A-B, or in alternative embodiments, the annular rib 550 may be located adjacent the outer edge of the nontransmissive portion 540. The annular rib 550 can used to treat myopia, for example, by locating the annular rib 550 on an outer portion of the nontransmissive region 540. The annular rib 550 can also be used to treat hyperopia, for example, by locating the annular rib 550 on an inner portion of the nontransmissive region 540. In certain embodiments, the outer portion is a portion of the nontransmissive region 540 that is more than half way from the inner periphery to the outer periphery of the nontransmissive portion 540, and the inner portion is a portion of the nontransmissive region 540 that is less than half way from the inner periphery to the outer periphery of the nontransmissive portion 540. By adjusting the diameter of the annular rib 550, the amount of corneal flattening, and thus the amount of refractive correction, can be adjusted. In addition, the thickness of the annular rib 550 can be varied to provide refractive correction. In certain embodiments, the annular rib 550 can be between about 150-450 microns thick or alternatively, between about 50-250 microns thick. In some embodiments, the rib(s) can be used in conjunction with the shape and/or thickness of the nontransmissive region 540 to produce a shape change in the cornea.

In certain embodiments, one or more ribs 552a-d can be located radially around the nontransmissive portion 540 to create a steepening of the cornea when the implant is positioned therein and thus provide correction for hyperopia, as shown in FIGS. 18C-D. In certain embodiments, the one or more ribs 452a-d can extend substantially from the inner edge to the outer edge of the nontransmissive portion 540. In alternative embodiments, the one or more ribs 552a-d can extend only partially from the inner edge of the nontransmissive portion 540. The number of ribs 552, spacing between the ribs 552 and thickness of the ribs 552 can be varied to provide adjustments to the curvature of the cornea for correcting the refractive error in the patient's eye. In certain embodiments, the ribs 552 are relatively thinner for a first portion of each of the ribs 552 than a second portion of each of the ribs 552. In certain embodiments, the first portion of each of the ribs 552 is relatively further from the outer edge of the nontransmissive portion 540 than the second portion of each of the ribs 552 to correct myopia. In other embodiments, the second portion of each of the ribs 552 is relatively further from the outer edge of the nontransmissive portion 540 than the first portion of each of the ribs 552 to correct hyperpia. In certain embodiments, the nontransmissive portion has at least one rib, at least two ribs, at least three ribs, at least four ribs, etc. that radially extending from the inner edge of the nontransmissive portion 540. The ribs 552 may be evenly spaced around the nontransmissive portion 540, or alternatively, the spacing between the ribs 552 may be varied to provided the desired corneal steepening. For example, the spacing and thickness of ribs can be adjusted to correct astigmatism. In certain embodiments, the ribs may have a thickness of between about 150-450 microns or alternatively, between about 50-250 microns.

While the above is a complete description of the preferred embodiments of the invention, various alternatives, modifications, and equivalents may be used. Also, elements or steps from one embodiment can be readily recombined with one or more elements or steps from other embodiments. Therefore, the above description should not be taken as limiting the scope of the invention which is defined by the appended claims.

1. An implant for positioning across an optical axis of a patient's eye, comprising:
an implant body, having a first zone for alignment with the optical axis the first zone comprising a first material and having a first transmissivity, and a second zone, comprising a second material and having a second, lower transmissivity, the second zone at least partially surrounding the first zone;
wherein the first zone comprises a water content of at least about 25% when immersed in normal saline at STP, and the second zone has a water content of less than about 10% when immersed in normal saline at STP.

2. An implant as in claim 1, wherein the water content of the first zone is at least about 30% and no more than about 55%.

3. (canceled)

4. (canceled)

5. An implant as in claim 1, wherein the second zone substantially surrounds the first zone.

6. An implant as in claim 1, wherein the second zone has a transmission of visible light of no more than about 15% of light in the visible range.

7. An implant as in claim 6, wherein the first zone has a transmission of visible light of at least about 85% of light in the visible range.

8. An implant as in claim 1, wherein the second zone has an anterior surface and a posterior surface and wherein the second zone comprises a plurality of randomly located recesses extending from at least one of said anterior and posterior surfaces.

9. An implant as in claim 8, wherein the second zone has substantially no water content.

10. (canceled)

11. An implant as in claim 8, wherein the plurality of randomly located recesses area configured to permit nutrient flow between a first corneal layer and a second corneal layer when the implant is implanted between said first and second corneal layers.

12. (canceled)

13. (canceled)

14. An implant as in claim 8, further comprising at least one non-randomly formed recess in said second zone, said non-randomly formed recess positioned in a location that maintains at least one performance characteristic of the mask.

15. An implant as in claim 1, wherein said first zone has a transverse dimension of at least about 2.5 mm.

16. (canceled)

17. An implant as in claim 1, wherein the first zone has an index of refraction substantially different from the cornea for providing refractive correction.

18. A corneal implant adapted for positioning between first and second layers of a cornea, comprising:
an annular mask portion having a transmission of light in the visible range of no more than about 20%; and
a central lens portion having a transmission of light in the visible range of at least about 80%;
wherein the lens portion has a water content of at least about 25% and the mask portion has a water content of no more than about 10% when immersed in normal saline at equilibrium at STP.

19. An implant as in claim 18, wherein said central lens portion has a transverse dimension of between about 2.5 mm-3.0 mm.

20. An implant as in claim 18, wherein said central lens portion has a transverse dimension greater than that which would produce a pinhole effect.

21. An implant as in claim 18, wherein the central lens portion has an index of refraction substantially different from the cornea for providing refractive correction.

22. An implant as in claim 18, wherein the annular mask substantially surrounds the central lens portion.

23. An implant as in claim 22, wherein the annular mask has an inner periphery and an outer periphery, said inner periphery adjacent said central lens portion, and wherein said annular mask has non-uniform thickness, said thickness decreasing from said inner periphery towards said outer periphery.

24. An implant as in claim 18, wherein the annular mask has an anterior surface and a posterior surface and wherein the annular mask comprises a plurality of randomly located recesses extending from at least one of said anterior and posterior surfaces.

25. An implant as in claim 24, wherein the plurality of randomly located recesses extend from the anterior surface through the posterior surface.

26. An implant as in claim 24, wherein the plurality of randomly located recesses area configured to permit nutrient flow between the first and second corneal layers when the implant is implanted.

27. (canceled)

28. (canceled)

29. An implant as in claim 24, wherein said plurality of recesses are configured such that when the implant is when implanted between the first and second corneal layers the recesses releasably draw in a portion of adjacent corneal tissue.

30. An implant as in claim 24, further comprising at least one non-randomly formed recess in said annular mask, said non-randomly formed recess positioned in a location that maintains at least one performance characteristic of the mask.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (Canceled)

47. (Canceled)

48. (Canceled)

49. (Canceled)

50. (Canceled)

51. (Canceled)

52. A method of treating a patient, comprising the steps of:
providing an ocular device comprising an annular mask portion having a transmission of light in the visible range of no more than about 20%; and a central lens portion having a transmission light in the visible range of at least about 80%; wherein the lens portion has a water content of at least about 25% and the mask portion has a water content of no more than about 10% when immersed in normal saline at equilibrium at STP; and
positioning the ocular device such that an optical axis of the patient intersects the central lens portion.

53. A method as in claim 52, wherein the positioning step comprises positioning the ocular device on an anterior surface of a cornea.

54. A method as in claim 52, wherein the positioning step comprises positioning the ocular device in between a first corneal layer and a second corneal layer.

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. A corneal implant adapted for positioning between first and second layers of a cornea, comprising:
an annular mask portion having a transmission in the visible range of no more than about 20%; and
a central lens portion having a transmission in the visible range of at least about 80%;
wherein the normalized expansion ratio of the lens to the mask in an aqueous environment is at least about 3:1.

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. A corneal implant adapted for implantation between layers of a cornea to focus an image on a retina of an eye, comprising:
a lens body having anterior and posterior surfaces and an outer circumference, said lens body comprising:
a clear, central region capable of refracting light to compensate for a refractive error of an eye; and
an annular nontransmissive region comprising a plurality of holes and extending from the outer circumference of the lens body to the clear central portion, said nontransmissive region extending over a minority of the surface area of the implant;
wherein said anterior and posterior surfaces are configured to abut adjacent layers of the cornea.

93. The corneal implant of claim 92, wherein said central region having a transverse dimension between about 2.5 mm and about 3.0 mm

94. The corneal implant of claim 92, wherein said central region has an optical power for providing refractive correction.

95. The corneal implant of claim 92, wherein the outer circumference comprises a transverse dimension between about 3.8 mm and about 4 mm.

96. The corneal implant of claim 92, wherein the lens body has a thickness of less than about 0.4 mm.

97. The corneal implant of claim 92, wherein the thickness of the lens body decreases toward said outer perimeter.

98. The corneal implant of claim 92, wherein the holes extend from the anterior surface through the posterior surface.

99. The corneal implant of claim 98, wherein said holes area configured to permit nutrient flow between layers of corneal tissue when the implant is implanted in a cornea.

100. (canceled)

101. (canceled)

102. The corneal implant of claim 98, wherein said plurality of recesses are configured to releasably draw in a portion of adjacent corneal tissue when implanted between said corneal layers.

103. (canceled)

104. (canceled)

105. (canceled)

106. (canceled)

107. (canceled)

108. (canceled)

109. (canceled)
